(19) United States
(12) Patent Application Publication
(10) Pub. No.: US 2026/0269008 A1
(43) Pub. Date: Sep. 10, 2026

(54) Systems and Methods for Sequencing and Analysis of Nucleic Acid Diversity

(71) Applicant: The Board of Trustees of the Leland Stanford Junior University, Stanford, CA (US)

(72) Inventors: Julia Salzman, Palo Alto, CA (US); Kaitlin Chaung, Stanford, CA (US); Tavor Baharav, Palo Alto, CA (US)

(73) Assignee: The Board of Trustees of the Leland Stanford Junior University, Stanford, CA (US)

(21) Appl. No.: 18/875,657

(22) PCT Filed: Jun. 14, 2023
(86) PCT No.: PCT/US2023/068448

§ 371 Date: Dec. 16, 2024

Related U.S. Application Data
(60) Provisional application No. 63/366,381, filed on Jun. 14, 2022.
(60) Provisional application No. 63/366,444, filed on Jun. 15, 2022.

(51) Int. Cl. G16B 30/00

(52) U.S. Cl. CPC G16B 30/00

(57) ABSTRACT

Systems and methods for assessment of sequence and molecule diversity are provided. In the realm of nucleic acids, sequencing results can be analyzed for diversity without alignment of sequencing reads to a reference sequence. The sequencing reads are utilized within a statistical framework to identify sequence anchors and sequence targets. The diversity among the sequence targets can be assessed for each sequence anchor.

### CROSS REFERENCE TO RELATED APPLICATIONS

This application claims priority to U.S. Provisional Application Ser. No. 63/366,381, entitled “Reference Free, Ultra Fast Valid Statistical Inference for Sequencing Data and Biological Sequences,” filed Jun. 14, 2022, and to U.S. Provisional Application Ser. No. 63/366,444, entitled “Reference Free, Ultra Fast Valid Statistical Inference for Sequencing Data and Biological Sequences,” filed Jun. 15, 2022, the disclosures of which are each incorporated herein by reference in their entirety.

### STATEMENT REGARDING FEDERALLY SPONSORED RESEARCH OR DEVELOPMENT

This invention was made with Government support under contract GM139517 awarded by the National Institutes of Health. The Government has certain rights in the invention.

### SEQUENCE LISTING

The instant application contains a Sequence Listing which has been submitted electronically in ASCII format and is hereby incorporated by reference in its entirety. The ASCII copy was created on Jun. 14, 2023, is named 08007PCT.xml, and is 6 KB in size.

### TECHNICAL FIELD

The disclosure provides description of systems and methods for sequencing and assessment of nucleic acid samples in which diversity of nucleic acids can be assessed without a reference sequence.

### BACKGROUND

Nucleic acid sequence diversification—mutation, reassortment or rearrangement of nucleic acids—is fundamental to evolution and adaptation across the tree of life. Diversification of pathogen genomes enables host range expansion, and as such host-interacting genes are under intense selective pressure. Sequence diversity in these genes is sample-dependent in hosts and in time as dominant strains emerge. In jawed vertebrates, V(D)J recombination and somatic hypermutation generate sequence diversity during the adaptive immune response that varies across cells, and is thus sample-dependent. Sequence diversification in the transcriptome takes the form of regulated RNA-isoform expression. This diversification enables varied phenotypes from the same reference genome including expression programs that enable cell specialization: cell-type specific splicing yields sample-dependent sequence diversity, samples being cells or cell-types. These examples are snapshots of sequence diversification which is core to wide-ranging biological functions, including adaptation, with applications in disparate fields ranging from plant biology to ecological metagenomics, among others.

### SUMMARY

In an aspect, a method of nucleic acid sequencing comprises calling significant anchor sequences from sequencing reads of a sequencing result of a nucleic acid sample. For an anchor sequence, sequence targets of that anchor can be assessed to determine sequence diversity.

In some embodiments, a computational method is for detecting nucleic acid diversity in a nucleic acid sequencing result. The method comprises retrieving a nucleic acid sequencing result of a plurality of nucleic acid samples utilizing a computational processing system. The nucleic acid sequencing result comprises a set of sequencing reads for each nucleic acid sample. The method comprises calling a set of anchor sequences utilizing the computational processing system. Each anchor sequence of the set is a contiguous sequence that is homologous across a subset of sequencing reads of at least one nucleic acid sample. The method comprises identifying a target sequence that is downstream at a defined distance from the anchor sequence of its sequencing read for each sequencing read of the subset of sequencing reads for each anchor sequence utilizing the computational processing system. The method comprises counting a number of unique anchor sequences and the number of targets for each unique anchor sequence for each sample utilizing the computational processing system. The method comprises determining a nucleic acid sequence diversity for each sample utilizing the computational processing system. The nucleic acid sequence diversity for each sample is determined utilizing the number of unique anchor sequences and the number of target sequences for each unique anchor sequence within a statistical test that is performed with a closed-form solution to calculate p-values to find anchors with significant sample-dependent target count distribution.

In some embodiments, each anchor sequence is defined as a sequence a where, given observing a in a sequence read, the conditional distribution of observing a target sequence t a distance R downstream of a is sample-dependent.

In some embodiments, each anchor sequence is set to a certain number of nucleotides.

In some embodiments, the certain number of nucleotides is between 3 nucleotides and 10 nucleotides.

In some embodiments, each anchor sequence is an adjacent, disjoint sequence.

In some embodiments, each anchor sequence is a tiled sequence that begins at a fixed step size.

In some embodiments, calling a set of anchor sequences comprises calling only anchors having a total count greater than threshold across all samples.

In some embodiments, the threshold is 50 total counts.

In some embodiments, calling a set of anchor sequences comprises discarding anchors that only appear in a number of samples below a threshold.

In some embodiments, the threshold is 2 samples.

In some embodiments, calling a set of anchor sequences comprises discarding anchor and sample pairs in which a number of sequence reads with anchors within the sample is below a threshold.

In some embodiments, the threshold is 6 reads with an anchor within the sample.

In some embodiments, the sequencing reads for each sample comprises at least 1,000 sequence reads.

In some embodiments, calling the set of anchor sequences comprises analyzing each sequencing read of each sample.

In some embodiments, each sequencing anchor of the set is a contiguous sequence that is homologous across a subset of sequencing reads of at least two nucleic acid samples.

In some embodiments, the method further comprises sequencing the plurality of nucleic acid samples to yield the nucleic acid sequencing result utilizing a high-throughput sequencer.

In some embodiments, the target sequences for each unique anchor sequence is utilized to generate a consensus target sequence.

In some embodiments, the target sequences for each unique anchor sequence is utilized to group target sequences via a homology branching method.

In some embodiments, the target sequences for each unique anchor sequence is utilized to lexicographically sort the target sequences.

In some embodiments, the method further comprises classifying at least one unique anchor sequence based on a sequence divergence of its target sequences utilizing the computational processing system.

In some embodiments, the classification is one of: nucleotide polymorphism, classical splice site, internal splice site, 3′UTR, centromere, or repeat.

In some embodiments, the method further comprises comprising aligning at least one unique anchor sequence with a reference to provide context utilizing the computational processing system.

In some embodiments, the method further comprises decoding at least one unique anchor sequence to yield an amino acid sequence utilizing the computational processing system.

In some embodiments, the plurality of nucleic acid samples comprises one of: a viral genome sample for assessing viral strains, a microbiome sample for assessing microbial genera or species diversity, a cancer sample for assessing clonal diversity, an expressed transcript sample for assessing paralogous gene expression, an expressed transcript sample for assessing alternative splicing events, an immune cell sample for assessing adaptive immunity recombination, or a genomic sample for assessing transposon activity.

### BRIEF DESCRIPTION OF THE DRAWINGS

The description and claims will be more fully understood with reference to the following figures and data graphs, which are presented as exemplary embodiments and should not be construed as a complete recitation of the scope of the disclosure.

FIG. 1 provides an example of a method to analyze sequence diversity of a sequencing result.

FIG. 2 provides an example of a computational processing system for assessing sequence diversity in sequencing results.

FIGS. 3A to 3E provide an overview of sample-dependent sequence diversification. FIG. 3A provides biological generality of sample-dependent sequence diversification. The study of sample-dependent sequence diversification unifies problems in disparate areas of genomics which are currently studied with application-specific models and algorithms. Viral genome mutations, alternative splicing, and V(D)J recombination all fit under this framework, where sequence diversification depends on the sample (through cell-type or infection strain type). Myriad problems in plant genomics, metagenomics and biological adaptation are subsumed by this framework. FIG. 3B provides an overview of the NOMAD pipeline. NOMAD takes as input raw FASTQ files for any number of samples>1 and processes them in parallel, counting (anchor, target) pairs per sample. NOMAD performs inference on these aggregated counts, outputting statistically significant anchors. For each significant anchor, a denoised per-sample consensus sequence is built (FIG. 3D). NOMAD also enables optional reference-based post-facto analysis. If a reference genome is available, NOMAD can align the consensus sequences to the reference, enabling denoised downstream analysis (e.g. SNPs, indels, or splice calls). In silico translation of consensuses can optionally be used to study relationships of anchors to protein domains by mapping to databases such as Pfam. FIG. 3C provides an overview of NOMAD compared to existing workflows. Existing workflows discard low-quality reads during FASTQ processing and alignment, only then performing statistical testing after algorithmic bias is introduced; p-values are then not unconditionally valid. Further, for every desired inferential task, a different inference pipeline must be used. NOMAD performs direct statistical inference on raw FASTQ reads, bypassing alignment and enabling data-scientifically driven discovery. Due to its generality, NOMAD can simultaneously detect myriad biological examples of sample-dependent sequence diversification. FIG. 3D provides NOMAD consensus building. NOMAD constructs a per-sample consensus sequence for every significant anchor by taking all reads in which the anchor (blue) appears, and recording plurality votes for each nucleotide, denoising reads while preserving the true variant; sequencing errors in red and biological mutations in purple. Existing approaches require alignment of all reads to a reference prior to error correction, requiring orders of magnitude more computation, discarding reads in both processing and alignment, and potentially making erroneous alignments due to sequencing error. They further require inferential steps, e.g. to detect if there is a SNP or alternatively spliced variant. FIG. 3E provides an example construction of NOMAD anchor, target pairs. A stylized expository example of viral surveillance: 4 individuals A-D are infected with one of two variants (orange and purple), differing by a single basepair (orange and purple). NOMAD anchor k-mers are blue (k=4), followed by a lookahead distance of L=2, and the corresponding k-mer targets. Given sequencing reads from the 4 individuals as shown, NOMAD generates a target by sample contingency table for this blue anchor, and computes a p-value to test if this anchor has sample-dependent sequence diversity.

FIGS. 4A and 4B provide NOMAD analysis of SARS-CoV-2 data. FIG. 4A provides stylized example representing NOMAD workflow for viral data. Patients with varying viral strains are sampled; two representative strains with differentiating mutations are depicted in orange and purple. NOMAD is run on raw FASTQs generated from sequencing patient samples. Significant anchors are called without a reference genome or clinical metadata. Optional post-facto analysis quantifies domain enrichment via in silico translation of consensus sequences derived from NOMAD-called anchors versus controls. Consensuses can also be used to call variants de novo and can be compared to annotated variants e.g. in SARS-CoV-2, Omicron. FIG. 4B provides NOMAD consensuses identify variants of concern de novo. Examples of NOMAD-detected anchors in SARS-CoV2 (France data). Scatterplots (left) show the fraction of each sample's observed fraction of target 1 (the most abundant target) for three representative anchors, binomial confidence intervals: (0.01,0.99), p=empirical fraction occurrence of target 1. y-axis shows histogram of the fraction occurrence of target 1. BLAT shows single nucleotide mutations match known Omicron mutations. Binomial p-values of 6.8E-8, 3.1E-7, and 6.7E-15 respectively. The anchor in (top) maps to the coronavirus membrane protein; anchors in (middle and bottom) map to the spike protein. One sample (out of 26) depicted in the bottom plot has a consensus mapping perfectly to the Wuhan reference; 3 other consensuses contain annotated Omicron mutations, some designated as VOC in May of 2022, 3 months after these samples were collected.

FIGS. 5A to 5D provide detection of differentially regulated alternative splicing and isoforms from single cell RNA-seq. FIG. 5A provides stylized diagram depicting differentially regulated alternative splicing detection with 3 exons and 2 isoforms with NOMAD. Isoform 1 consists of exon 1 and exon 2, and is predominantly expressed in cell A. Isoform 2 consists of exon 1 and exon 3 and is primarily expressed in cell B. An anchor sequence in exon 1, then generates target sequences in exon 2 or exon 3. Counts are used to generate a contingency table, and NOMAD's statistical inference detects this differentially regulated alternative splicing. FIG. 5B provides detection of differential regulation of MYL12A/B isoforms. (top-left) Shared anchor (q-value 2.5E-8, donor 1, 2.3E-42 for donor 2) highlighted, maps post fact to both MYL12 isoforms, highlighting the power of NOMAD inference: MYL12A and MYL12B isoforms share >95% nucleotide identity in coding regions. (bottom-left) NOMAD's approach automatically detects target and consensus sequences that unambiguously distinguish the two isoforms. (right) In both donors, NOMAD reveals differential regulation of MYL12A and MYL12B in capillary cells (MYL12A dominant) and macrophages (MYL12B dominant). FIG. 5C provides that NOMAD identifies single-cell-type regulated expression of HLA-DRB1 alleles. NOMAD shared anchor, q-value of 4.0E-10 for donor 1, 1.2E-4 for donor 2. Scatter plots show cell-type regulation of different HLA-DRB1 alleles not explained by a null binomial sampling model p<2E-16 for donor 1, 5.6E-8 for donor 2, finite sample confidence intervals depicted in gray. Each (donor, cell-type) pair has a dominant target, per-cell fractions represented as “fraction target 1” in scatterplots, and a dominant consensus mapping to the HLA-DRB1 3′ UTR (multiway alignment); donor 1 capillary consensus contains an insertion and deletion. FIG. 5D provides cell-type specific splicing of HLA-DPA1 in capillary versus macrophage cells. Anchor q-value: 7.9E-22. Detected targets are consistent with macrophages exclusively expressing the short splice isoform which excises a portion of the ORF and changes the 3′ UTR compared to the dominant splice isoform in capillary cells; splice variants found de novo by NOMAD consensuses. Binomial hypothesis test as in D for cell-type target expression depicted in scatter plots (binomial p<2.8E-14).

FIGS. 6A to 6D provide unsupervised identification of V(D)J recombination in human and lemur immune cells. FIG. 6A provides stylized diagram depicting NOMAD detection of V(D)J recombination, with example variable regions in the heavy chain. An anchor sequence in the constant region, generates target sequences during V(D)J recombination, in which immunoglobulins may receive different gene segments during rearrangement. NOMAD is able to rediscover and detect these recombination events by prioritizing sample-specific TCR and BCR variants. FIG. 6B that NOMAD detects cell-type and allele-specific expression of HLA-B and HLA-B alleles de novo. NOMAD-annotated anchors are enriched in HLA-B (top). Sample scatterplot (middle) The figure shows that T cells have allelic-specific expression of HLA-B, not explicable by low sampling depth (binomial test, p<4.6E-24). HLA-B sequence variants are identified de novo by the consensus approach (bottom), including allele-specific expression of two HLA-B variants, one annotated in the genome reference, the other with 5 SNPs coinciding with annotated SNPs. FIG. 6C provides that NOMAD detects combinatorial expression of T cell receptors in immune cells de novo. In human T cells (right), a NOMAD anchor was identified in the TRVB7-9 gene, and two example consensuses which map to disjoint J segments, TRBJ1-2 and TRBJ2-7. Histograms of this anchor depict combinatorial single-cell (columns) by target (row) expression of targets detected by NOMAD. Histogram for lemur T cells depicted similarly; lemur T cell anchor maps to the human gene TBC1D14. FIG. 6D provides that NOMAD analysis of lemur and human B (left) and T (right) cells recovers B, T cell receptors and HLA loci as most densely hit loci. Human genes are depicted as triangles; lemur as circles. Post facto alignments show variable regions in the kappa light chain in human B cells are most densely hit by NOMAD anchors and absent from controls; in T cells, the HLA loci and TRB including its constant and variable region are most densely hit, which are absent from controls. x-axis indicates the fraction of the 1000 control anchors (most abundant anchors) that map to the named transcript, y-axis indicates the fraction of NOMAD's 1000 most significant anchors that map to the named transcript. Each inset depicts anchor density alignment in the IGKV region (left) and HLA-B in CD4+ T cells (top right) and TRBC-2 (bottom right), showing these regions are densely hit.

FIGS. 7A and 7B provide a general reference-independent alignment-free approach with diverse RNA-Seq analysis applications. FIG. 7A: Anchors called by NOMAD+ are extended through a local assembly approach to generate “compactors” that are used in the subsequent classification step for inference and downstream analysis. FIG. 7B: Compactors for the called anchors by NOMAD+ are classified into multiple biologically relevant groups: splicing, base pair change, internal splicing, 3′UTR, centromere, and repeats. The softclipped bases for the compactors that have been aligned to the genome through softclipping are realigned to the genome using Bowtie aligner and are used to find compactors with evidence for circular RNA, gene fusions, strandcrosses. Compactors that fail to map are annotated by the identity of other compactors in its family, called annotation by association.

FIGS. 8A to 8E provide that NOMAD+ enables de-novo analysis of alternative splicing in single cells. FIG. 8A: Dot plot of the number of distinct anchors classified as splicing in each pair of donor and tissue. >55% of the splicing anchors found in each donor and tissue involve an annotated alternative splicing event. FIG. 8B: Upset plots comparing the concordance of the significant splicing genes called by NOMAD+ and SpliZ in two replicates of lung (left) and blood (right). NOMAD+ achieves higher concordance of the called genes in the same tissue from different donors compared to SpliZ despite not using cell identity, which have different distributions and or composition in the two donors. FIG. 8C: The plot shows that the compartment-specific alternative splicing of GAS5 is reproducible in muscle cells from 3 different donors. The error bar for each dot shows the 95% binomial confidence interval for each isoform fraction. In all 3 donors, CD8+, alpha-beta t cells possess higher inclusion rate for the isoform with shorter intron. FIG. 8D: Heatmap showing the fraction of eight CD47 isoforms detected for an anchor of this gene in each single cell across 10 donors and 14 tissues. Cells with >5 reads are included and horizontal side bars show the donor, tissue, and compartment of each cell. Cells are sorted based on hierarchical clustering applied directory to the heatmap. NOMAD+ detects extensive expression variation, including novel isoforms. Among other detected CD47 isoforms is a novel isoform (shown in yellow) with annotated junctions (chr3:110767091:110771730-chr3:110771761:110775311). FIG. 8E: Extensive alternative splicing of RPS24 for an anchor with 4 different splicing isoforms. The alternative splicing involves inclusion/exclusion of two cassette exons in ultraconserved regions, including a microexon of 3 bps. NOMAD+ detected a novel isoform which includes only the micro exon detected. The multiway alignment confirmed the inclusion of the microexon while both STAR and BLAT were unable to detect the microexon. The heatmap shows the fraction of these 4 different isoforms across lung cells from donor 2, which also shows the compartment-specific alternative splicing where the isoform with both exons included is predominantly expressed in epithelial cells.

FIGS. 9A to 9C provide that global trends of compactor diversity reveal V(D)J recombination has highest levels of transcriptome diversity and BraCeR comparison. FIGS. 9A and 9B: Comparison of NOMAD+ and BraCeR for donor 2 and donor 7 spleen among cells analyzed by both algorithms. Each dot represents a cell which is BraCeR+ if a BraCeR contig was called, and NOMAD+ if an IG-compactor was found with stringent filters (Methods) and expression over 5 counts. The value on the x-axis is the expression of the maximally expressed IG-compactor found in that cell. NOMAD+ largely agrees with BraCeR but both (i) missed calls BraCeR makes such as in donor 7 memory B cells, and (ii) finds IG events in B cells that BraCeR does not, such as donor 2 plasma and memory B cells. FIG. 9C: Fraction of cells run through both BraCeR and NOMAD+ where at least one IG-compactor has Hamming distance less than threshold to at least one BraCeR contig in the same cell.

FIGS. 10A to 10F provide NOMAD2 pipeline outline and resource consumption. FIG. 10A: Outline of the NOMAD2 pipeline for toy data (2 input samples, anchor length 3, gap length 0, target length 3. FIG. 10B: Change in anchor, gap, target length has a small impact on computation time, RAM usage, and the number of detected significant anchors. FIG. 10C: Memory requirements depend on the stage and dataset. FIG. 10D: Increase in the number of threads speeds up computation, but also increases RAM requirements. FIG. 10E: Time and RAM usage increases linearly with an increasing number of input samples. FIG. 10F: Run time increases linearly with an increasing number of input reads for a given number of samples, yet memory saturates and does not exceed 15 GB.

FIGS. 11A to 11G provide that NOMAD2 enables rapid discovery at scale in scRNA-Seq and cancer cell lines. FIG. 11A: Bar plot showing the number of anchors detected by NOMAD2 in SS2 muscle scRNA-Seq for each mismatch category and number of mismatches (in total: 512 anchors from 303 genes). Consistent with the canonical RNA editing, G<->A category has the highest number of anchors for each number of mismatches. FIG. 11B: Among genes with >3 mismatches, AGO2, TDRP, and PECAM1, were found in the highest number of cells. For each gene, the multiple sequence alignment for the reference and the four most abundant extendors was depicted. The bar plots show the total read count for each extendor. Black arrows show the position of mismatches for each gene. FIG. 11C: NOMAD2 identified pervasive unannotated alternative splicing in SS2 muscle cells. Genes SNHG17, DIMT1, and CAMLG were the genes with unannotated alternative splicing involving one annotated and one unannotated junction found in the highest number of cells. Bar plots show the total read count for each junction. FIG. 11D: Simultaneous detection of mutations and a novel splice junction for PTEN (a known tumor suppressor) in CCLE. The annotated junction harbors two mutations (one internal and one at splice site) and was expressed specifically in upper aerodigestive carcinoma. FIG. 11E: NOMAD2 detected a cryptic exon for RAB36 in the intron between 7th and 8th exons which had higher expression than the canonical splicing in lymphoma cell lines. FIG. 11F: NOMAD2 detected a novel cryptic splicing for SMIM14 with cancer-type-specific expression in lymphoma and leukaemia cell lines. FIG. 11G: The anchor from SLC3A2 had the highest fraction of unaligned reads by STAR (for Extendor1) which was expressed solely in upper aerodigestive carcinoma and includes exonic sequence from SLC3A2 fused to PDE1C.

### DETAILED DESCRIPTION

Turning now to the drawings and data, systems and methods for performing sequencing and analysis of diverse nucleic acid samples are described in accordance with the various embodiments of the description. The systems and methods provide a statistical means for accurately assessing nucleic acid sequencing data without the use a reference sequence. The system and methods can discover the level of diversity within sequencing data.

Current practiced methods to detect sample-specific diversity are highly specialized to each application and lack conceptual unification, creating several issues. One issue is that biological inference is limited if a workflow is chosen that does not have power to detect significant signals in the data. For example, if the workflow does not map transposable element insertions, none will be found, as these variants are missing from references.

Another issue is that the first step in existing approaches almost always requires the use of references through alignment or their construction de novo. In human genomics, workflows beginning with reference alignment miss important variation absent from assemblies, even with pan-genomic approaches. For example, genetic variants associated with ancestry of under-studied populations are poorly represented in databases and result in health disparities. In disease genomics, sequences of pathogenic cells may be missing from reference genomes, or no reference genome exists, as in genetically unstable tumors. Many species do not have reference genomes even when they in principle could be attained due to logistical and computational overheads. In these cases, alignment-based methods (also referred to as “reference-first approaches”) approaches fail. In viral surveillance, reference-first approaches are even more problematic. Viral reference genomes cannot capture the complexity of viral quasispecies or the vast extent of polymorphism. New viral assemblies are constantly being added to reference databases. In the microbial world, pre-specifying a set of reference genomes is infeasible due to its inherent rapid genomic changes. References also cannot capture insertional diversity of mobile elements, which have significant phenotypic and clinical impact and are only partially cataloged in references.

Another issue is that current approaches have severe technical limitations. Statistical analysis in reference-first approaches is conditional on the output of the noisy alignment step, making it difficult or impossible to provide valid statistical significance levels in downstream analyses, such as differential testing or expression analysis. When available, computationally intensive resampling is required.

Biological studies today typically provide deep sampling of the nucleic acid composition, for example through RNA-seq or DNA-seq. In principle, this data provides an opportunity to identify sample-dependent sequence diversity with powerful and efficient statistical models. One example is sequencing of SARS-CoV-2: sequences in the spike glycoprotein are diversified as strains compete, for example during the emergence of the omicron variant. Patient samples collected in late 2021 include sampling of Delta and Omicron strains, thus containing sample-dependent sequence diversity in regions that differentiate the strains, including but not limited to ones in the spike protein coding region. As described in detail herein, statistical approaches are able to capture this diversity without a reference genome.

Here, systems and methods perform sequencing and analysis of nucleic acid samples comprising statistically assessing the diversity of nucleic acid sequences. In many embodiments, the systems and methods do not rely on a reference sequence to align sequence reads. In several embodiments, instead of utilizing a reference, the statistical method assesses the k-mers by identifying and grouping homologous sequences and further assessing the downstream sequence diversity.

Although nucleic acid sequence assessment is described throughout, it should be understood that the systems and methods can be applied to other specimens. For instance, protein sequence diversity and chemical formula diversity can be assessed utilizing the same principles of the statistical methods described herein.

Several embodiments of the systems and methods of the disclosure provide a means for improving the analysis of sequence diversity in a sequencing result of a sample. In many embodiments, the systems and methods do not require alignment of the sequencing reads to a reference sequence to call out polymorphisms and other variations in sequence. Instead, several embodiments of the systems and methods call significant “anchor sequences,” which is a string of a conserved homologous sequence among the sequence reads. A closed statistical operation can determine which anchor sequences are significant for further analysis.

In many embodiments, sequence reads are assessed to identify diverse “target sequences,” which are sequences downstream of the anchor. In some instances, a “gap sequence” may be between an anchor sequence and a target sequence. In several embodiments, k-mers (anchor+optional gap+target) are analyzed. Many different associations among the k-mers can be made, such as (for example) determining a consensus target sequence for an anchor, merging target sequences, and grouping of target sequences based on homology. Various analyses can be performed on k-mers, including (but not limited to) classification, annotation, alignment with references/databases, decoding of protein sequences, and assessing sequence diversity.

The various embodiments of the systems and methods can be performed on any nucleic acid sample, including DNA samples and RNA samples. Virtually any nucleic acid sample will have diversity of sequences and thus can be assessed by the systems and methods described herein. Some particular nucleic acid samples that may be of interest for assessing diversity include (but are not limited to) nucleic acids of: virus samples, microbiome samples, neoplastic/cancer samples, samples of B-cell/T-cell recombination, alternative splicing, paralog gene expression, transposon activity, cell-free sources, etc.

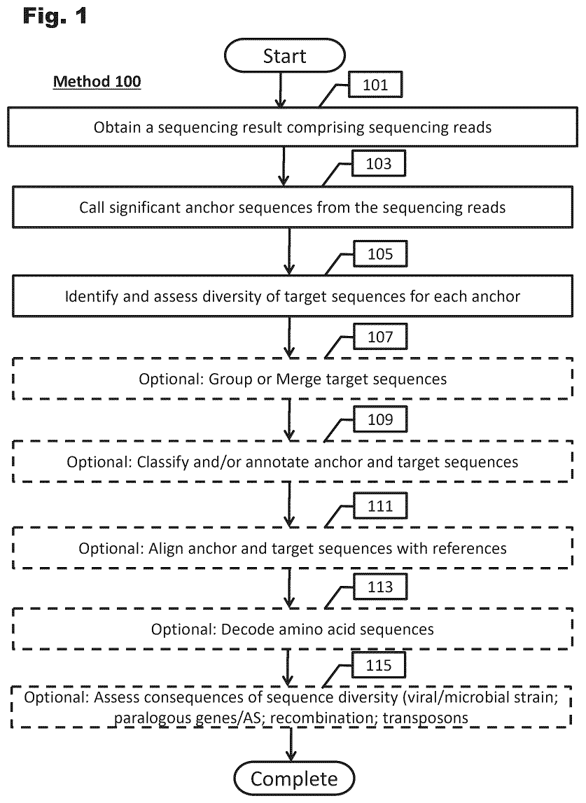

Provided in FIG. 1 is an example of a method to assess sequence diversity of a nucleic acid sample. Method 100 can begin by obtaining (101) a sequencing result comprising sequencing reads for a plurality of nucleic acid samples. Generally, high-throughput sequencing data can be analyzed in which the sequencing procedure yields a high number of sequencing reads.

Sources of nucleic acids for sequencing can be derived from any organism or any collection of organisms. DNA nucleic acid sources include (but are not limited to) ssDNA viral genomes, dsDNA viral genomes, microbial genomes, eukaryotic genomes, chromosomal DNA, circular DNA, and cell-free DNA. RNA nucleic acid sources include (but are not limited to) ssRNA viral genomes, dsRNA viral genomes, cellular transcript expression, ribosomal RNA, and cell-free RNA.

The source material can be sheared or digested into a collection of originating nucleic acid molecule fragments. In the case of cfDNA, the extracted and isolated cfDNA fragments can be utilized as originating nucleic acid molecule fragments. A collection of originating nucleic acid molecule fragments (e.g., cell-free nucleic acid molecules or sheared nucleic acid molecules) for library preparation can have greater than 10,000 originating nucleic acid molecule fragments, greater than 100,000 originating nucleic acid molecule fragments, greater than 1,000,000 originating nucleic acid molecule fragments, greater than 10,000,000 originating nucleic acid molecule fragments, greater than 100,000,000 originating nucleic acid molecule fragments, or greater than 1,000,000,000 originating nucleic acid molecule fragments.

The sequencing data comprising sequencing reads, as disclosed herein, can be obtained from one or more sequencing methods. Generally, a nucleic acid sample is prepared into a library and then sequenced using a high-throughput technique. The nucleic acid sample can be an RNA sample or a DNA sample. Libraries can be constructed by amplifying the source nucleic acids. RNA samples can be converted into cDNA utilizing a reverse-transcriptase technique. Adapters and/or barcodes can be added onto the nucleic acids of the library to assist in analysis of the sequencing results.

In some embodiments, the sequencing data can be obtained based on any sequencing method that utilizes adapters. Nucleic acid samples can be conjugated with one or more adapters (or adapter sequences) for recognizing (e.g., via hybridization) of the sample or any derivatives thereof (e.g., amplicons). In some examples, the nucleic acid samples can be tagged with a molecular barcode, which may be utilized as a unique molecular identifier. Alternatively, or in addition, the nucleic acid samples can be tagged with a sample barcode, which may be utilized as a sample identifier.

In some embodiments, sequencing library preparation may include nucleic acid amplification. Amplification can be performed by any appropriate means, including (but not limited to) polymerase chain reaction (PCR), whole genome amplification (WGA), in vitro transcription (IVT), or any combination of amplification techniques. In some implementations, a single or double stranded adapter is ligated to a nucleic acid molecule fragment and is then further amplified by PCR. In some embodiments, Adaptase (Swift Biosciences, Ann Arbor, MI) is used to simultaneously tail and ligate an adapter, the strand is then further amplified by PCR. In some embodiments, particular regions of interest are amplified.

In some implementations, a library is prepared for targeted sequencing. Target sequence involves the enrichment of regions of interest, which can be performed by amplification techniques and/or capture hybridization techniques. Hybridization involves utilizing nucleic acid probes to anneal and capture particular sequences. Capture hybridization can be performed before amplification. Targeted sequencing may be useful for sequencing particular sequences of interest. In some embodiments, exome sequencing (e.g., whole exome sequencing (WES) can be performed.

In several embodiments, sequencing of a library can be performed via a high-throughput sequencing technique, such as next-generation sequencing (NGS) (e.g., sequencing by synthesis). A high-throughput sequencing method can sequence simultaneously (or substantially simultaneously) at least about 10,000, at least about 100,000, at least about 1 million, at least about 10 million, at least about 100 million, at least about 1 billion, or more molecules of a nucleic acid library. Examples of high-throughput sequencing methods include (but are not limited to) massively parallel signature sequencing, polony sequencing, pyrosequencing, sequencing-by-synthesis, combinatorial probe anchor synthesis (cPAS), sequencing-by-ligation (e.g., sequencing by oligonucleotide ligation and detection (SOLiD) sequencing), semiconductor sequencing (e.g., Ion Torrent semiconductor sequencing), DNA nanoball sequencing, single-molecule sequencing, and sequencing-by-hybridization.

Upon sequencing, in accordance with several embodiments, the sequencing reads are utilized for further analysis. A sequencing read can be contiguous or paired-end. A sequencing read can be provided in any data format, such as (for example) FASTA and FASTQ. In various embodiments, the number of sequencing reads for each sample is at least about 1,000 reads, at least about 10,000 reads, at least about 100,000 reads, at least about 1 million, at least about 10 million reads, at least about 100 million reads, or at least about 1 billion reads.

Method 100 calls (103) anchor sequences from the sequencing reads. An anchor sequence is a contiguous sequence of nucleotides that is a subsequence of a sequence read. Anchor sequences that are determined to be significant are shared among a number of sequence reads and have a target sequence at a defined distance downstream of the anchor. In some embodiments, anchors are defined as sequences a where, given observing a in a sequence read, the conditional distribution of observing a target sequence ta distance R downstream of a is sample-dependent. Anchor sequences can be set to a certain number of nucleotides (e.g., between 3 and 10 nucleotides). Anchor sequences can be extracted as adjacent, disjoint sequences or as tiled sequences that begin at a fixed step size. In some implementations, to reduce the number of hypotheses tested and required to correct for, p-value calculation was used only for anchors having a count greater than a threshold (e.g., 50 total counts) across all samples. In some implementations, anchors are discarded that only appear in a number of samples below a threshold (e.g., in less than two samples). In some implementations, a sample and anchor pair is discarded in the number of sequence reads with anchors within the sample is below a threshold (e.g., below 6 reads with an anchor within the sample).

Method 100 identifies and assesses (105) diversity of target sequences for each anchor. In several embodiments, anchor and target sequences are counted for each sample. To generate counts, a contingency table or matrix can be generated containing the read counts for each target and each sample. Upon counting, in accordance with many embodiments, diversity is assessed using a statistical test that is performed with a closed-form solution to calculate p-values to find anchors with significant sample-dependent target count distribution. An example of a test statistic that can be utilized is described and validated within the Examples sections. In many embodiments, the statistical test is only performed on a subset of anchors of the data set (e.g., above a threshold, within more than one sample, etc.). Notably, sequence diversity is assessed without alignment to a reference sequence.

Upon assessment of sequence diversity, several further analyses and downstream assessments can be performed, including (but not limited to) grouping/merging (107) target sequences, classifying/annotating (109) anchor and target sequences, aligning (111) anchor and target sequences with references/databases, decoding (113) amino acid sequences and assessing homology, and assessing (115) consequences of sequence diversity in variety applications (e.g., viral/microbial strain diversity, clonal sequence diversity, expression of paralogous genes/alternatively spliced transcripts, recombination events, transposon activity, etc.).

Method 100 optionally groups or merges (107) target sequences. Various techniques of grouping and/or merging can be performed, including (but not limited to) yielding a consensus sequence, grouping target sequences via homology branching methods, and lexicographically sorting target sequences.

In some embodiments, a consensus target sequence is generated for a set of one or more anchors. Any of a number of methods to generate a consensus method can be utilized. In one example, for each nucleotide of the target, the consensus is determined by the plurality (i.e., most common) of all sequence reads sharing the same anchor. The consensus base and/or the fraction of the plurality can be recorded and stored. Consensus sequences can be utilized in a number of downstream discoveries in assessing divergence of sequences, including (but not limited to) identification of point mutations, alternative splice sites, recombination events, and sequence diversity.

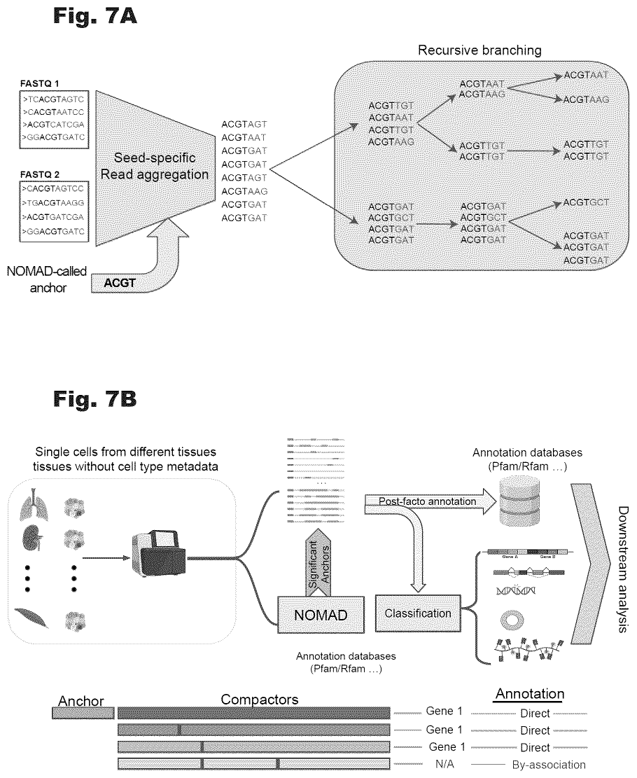

In some embodiments, target sequences are grouped based on homology using a branching method. Grouped targeted sequences can be referred to as “compactor sequences.” Any of a number of methods of grouping can be utilized. In one example, for each anchor, reads are assessed 5′ to 3′ and nucleotides at each position are examined to determine if the nucleotide variation is greater than threshold. For example, a compacter sequence can be yielded if a minimum number of reads present a particular nucleotide and the read number exceeds a percentage of reads on the current branch, this branch is kept and further assessed downstream. This rule is applied recursively, resulting in subsets of all anchor-reads each represented by a distinct compactor sequence (see FIG. 7A). Compactor sequences can be utilized in a number of downstream discoveries in assessing divergence of sequences, including (but not limited to) identification of point mutations, alternative splice sites, recombination events, and sequence diversity. In some implementations, only a subset of the most abundant compactor sequences is further assessed. Further references of target sequences in downstream analyses described herein should be interpreted to include compactor sequences.

In some embodiments, target sequences are lexicographically sorted, which can be done by a various computational applications. One example of an application that can be used is the KMC-tools. Lexicographical sorting results in adjacently grouping homologous target sequences, which can be merged across samples. In some embodiments, to reduce computation efforts, counts are updated upon merging and/or gaps between anchors and target sequences removed, reducing the amount of sequence data for p-value computation and downstream assessments. Further filters can be applied to reduce sequence data. Merged and concatenated anchor-target pairs can be referred to as extendor sequences, which can be utilized in a number of downstream discoveries in assessing divergence of sequences, including (but not limited to) identification of point mutations, alternative splice sites, recombination events, and sequence diversity. In some implementations, only a subset of the most abundant extendor sequences is further assessed. Further references of target sequences in downstream analyses described herein should be interpreted to include extendor sequences.

Method 100 optionally classifies and/or annotates (109) anchor and target sequences based on its sequence divergence. Examples of classifications and/or annotations include (but are not limited to) nucleotide polymorphism, classical splice site, internal splice site, 3′UTR, centromere, and repeat. In various implementations, a consensus target sequence, a compactor sequence, or an extender sequence is classified or annotated. Any of a number of methods to classify or annotate anchor and target sequences can be utilized. In one example, an anchor and target sequence is classified or annotated as a classical splice site if the target sequence maps to a canonical splice junction. If a canonical splice site is not present, an anchor and target sequence is classified or annotated as an internal splice site or base pair change according to the string edit distance between the diverging target sequences. An anchor and target sequence can be classified a 3′ UTRs, a centromere, or a repeat if it intersects with annotated database.

Distances between diverging target sequences can be computed utilizing any of a number of distance metrics. Examples include (but are not limited to) hamming distance and Levenstein distance. In an example, diverging target sequences with the same hamming and Levenstein distances are classified as mutations as this criterion indicates that only substitutions (i.e., nucleotide changes) can explain the difference between the compactors. Further, in this example, if the Levenstein operations sequence for diverging target sequences includes a number of consecutive insertions and deletions, the divergence is classified as internal splicing.

Method 100 optionally aligns (111) anchor and target sequences with references. A variety of references can be utilized, such as reference sequences or reference databases. Based on alignment of a reference, context can be provided to the sequence, such as determining if the sequence is intronic, exonic, associated with a gene, has a molecular function, or is a particular structure. For example, sequences can be aliened to databases of sequencing artifacts, transposable elements, and mobile genetic elements to determine if the sequence is related to any of these structures. Sequences can also be aligned to referential and annotated genomes, such as a reference human genome. Examples of references include (but are not limited to): UniVec, Illumina adapters, Escherichia phage phiX174, Rfam, Dfam, TnCentral, ACLAME, ICEberg, CRISPR direct repeats, ITSoneDB, ITS2, WBcel235, TAIR10, and grch38_1 kgmaj.

In some embodiments, a reference genomic sequence can be at least a portion of a nucleic acid sequence database (i.e., a reference genome), which database is assembled from genetic data and intended to represent the genome of a reference cohort. In some cases, a reference cohort can be a collection of individuals from a specific or varying genotype, haplotype, demographics, sex, nationality, age, ethnicity, relatives, physical condition (e.g., healthy or having been diagnosed to have the same or different condition, such as a specific type of cancer), or other groupings. A reference genomic sequence can be a mosaic (or a consensus sequence) of the genomes of two or more individuals. The reference genomic sequence can comprise at least a portion of a publicly available reference genome or a private reference genome. Non-limiting examples of a human reference genome include hg119, hg18, hg17, hg16, and hg38.

Method 100 optionally decodes (113) amino acid sequences, which can be used to assess protein sequence homology. Anchor and target sequences can be translated, assessing each open reading frame to obtain putative amino acid sequences. In some embodiments, these sequences are aligned with an annotated protein sequence data base to determine which protein or peptide is encoded by the nucleotide sequence. Any of a number of annotated protein databases can be utilized such as (for example) Pfam. Upon acquiring amino acid sequences, protein sequence homology between the divergent target sequences can be assessed.

Method 100 optionally assesses consequences of the sequence diversity. A number of downstream analyses can be performed. In some embodiments, the sequences are related to viral genome sample and the sequence diversity can provide detail on the strains of virus in the sample. Because no reference sequence is utilized for alignment, the statistical method employed here can detect viral strains that are undetectable utilizing the standard referential alignment analysis. Accordingly, this method can more efficiently identify novel viral strains as they emerge than the standard referential alignment methods.

In some embodiments, sequences are related to a microbiome sample and the sequence diversity can provide detail on the microbial genera and/or species of virus within the microbiome. This method can detect microbial genera and/or species diversity better than method that must rely a plurality of reference genomes for each microbial genera and/or species. This method can better identify microbial diversity (e.g., Shannon diversity index) and novel or rare microbial genera/species which can provide insights on host health.

In some embodiments, sequences are related to a neoplastic or cancer sample and the sequence diversity can provide detail on clonal diversity of the neoplasm or cancer. Understanding the diversity of clones within a neoplasm or cancer can better inform cancer aggressiveness, survivability, and treatment options.

In some embodiments, sequences are related to expressed transcript samples and the sequence diversity can provide insight on paralogous gene and/or alternatively spliced transcript levels. Use of a traditional referential alignment method can fail to detect the expression of a paralogous gene or an alternative spiced transcript, especially when expressed at lower levels. This method better identifies these overlooked transcripts, which can potentially be utilized as a biomarker or better assess a particular phenotype.

In some embodiments, sample sequences are related to gene recombination and the sequence diversity can provide insight on the diversity and specificity of particular recombination events. For example, an immune cell sample can be utilized to assess adaptive immunity recombination (e.g., V(D)J recombination) associated with B-cell and T-cell maturation, which can yield particular humoral and T-cell responses. This method can better assess adaptive immunity recombination which can lead insight into a patient's immunity status or better determine potential immunotherapies.

In some embodiments, sequences are related to transposition and the sequence diversity can provide insight on transposon activity. Transposition events can affect gene expression and yield a mosaicism of cells with a sample. Transposition events have been found to be important in cancer, brain development, and plant biology. Traditional referential annotation analysis disregards transposon sequences as it is difficult to align such sequences with the number of repeats within the genome. This method provides an opportunity to better asses transposon activity to better determine its effect on the host genome.

While specific examples of methods for assessing diversity of nucleic acid sequencing results are described above, one of ordinary skill in the art can appreciate that various steps of the method can be performed in different orders and that certain steps may be optional according to some embodiments. Further, various steps of the method could be modified as appropriate to the requirements of specific applications.

### Computational Processing System

A computational processing system to assess diversity of nucleic acid sequencing results in accordance with the various methods of the disclosure typically utilizes a processing system including one or more of a CPU, GPU and/or neural processing engine. In a number of implementations, sequencing results are processed and assessed to detect diversity among sequencing reads using a computational processing system. In some implementations, the computational processing system is housed within a computing device associated with sequencer. In some implementations, the computational processing system is housed separately from the sequencer and receives the sequencing results. In certain embodiments, the computational processing system is implemented using a software application on a computing device such as (but not limited to) mobile phone, a tablet computer, a wearable device (e.g., watch), and/or portable computer. In certain embodiments, the computational processing system is a cloud system or otherwise transmits data via wired or wireless connections.

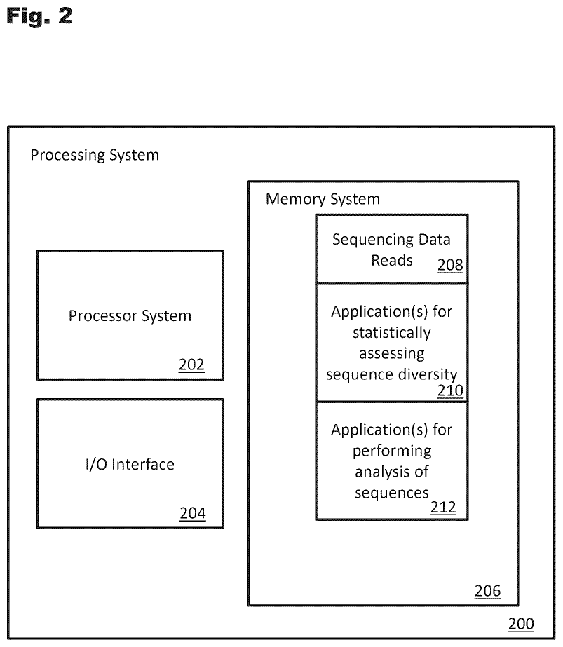

A computational processing system in accordance with various embodiments of the disclosure is illustrated in FIG. 2. The computational processing system 200 includes a processor system 202, an I/O interface 204, and a memory system 206. As can readily be appreciated, the processor system 202, I/O interface 204, and memory system 402 can be implemented using any of a variety of components appropriate to the requirements of specific applications including (but not limited to) CPUs, GPUs, ISPs, DSPs, wireless modems (e.g., WiFi, Bluetooth modems), serial interfaces, volatile memory (e.g., DRAM) and/or non-volatile memory (e.g., SRAM, and/or NAND Flash). The memory system is capable of storing a sequencing data reads 208, applications for applications for statistically assessing sequence diversity 210, and applications for performing analysis of sequences. The applications can be downloaded and/or stored in non-volatile memory. When executed, the applications for assessing sequence diversity or for performing sequence analysis are capable of configuring the processing system to implement computational processes including (but not limited to) the computational processes described above and/or combinations and/or modified versions of the computational processes described above. In several embodiments, the various applications 210 and 212 utilize the sequence data reads 208 to assess sequence diversity and analyses thereof.

While specific computational processing systems are described above with reference to FIG. 2, it should be readily appreciated that computational processes and/or other processes utilized in the provision of assessing modification status in sequencing results in accordance with various embodiments of the disclosure can be implemented on any of a variety of processing devices including combinations of processing devices. Accordingly, computational devices in accordance with the disclosure should be understood as not limited to specific computational processing systems, but can be implemented using any of the combinations of systems described herein and/or modified versions of the systems described herein to perform the processes, combinations of processes, and/or modified versions of the processes described herein.

### EXAMPLES

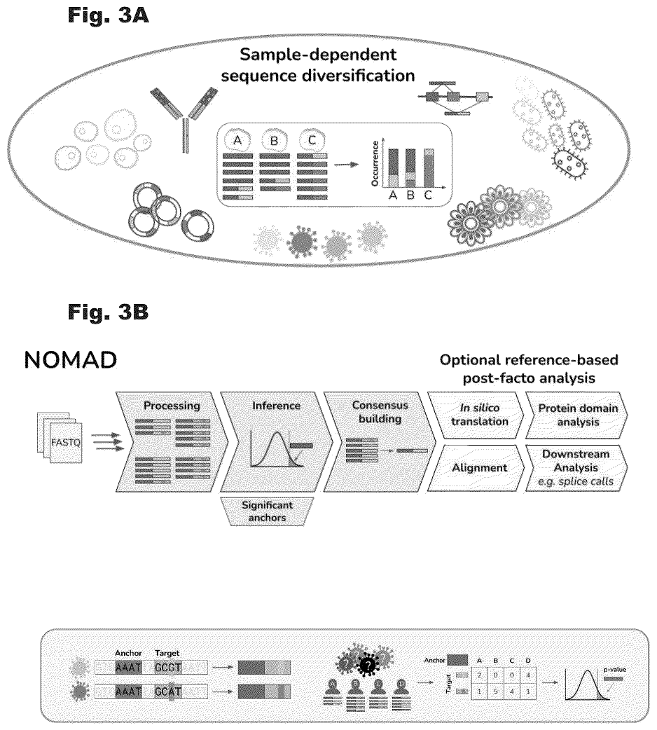

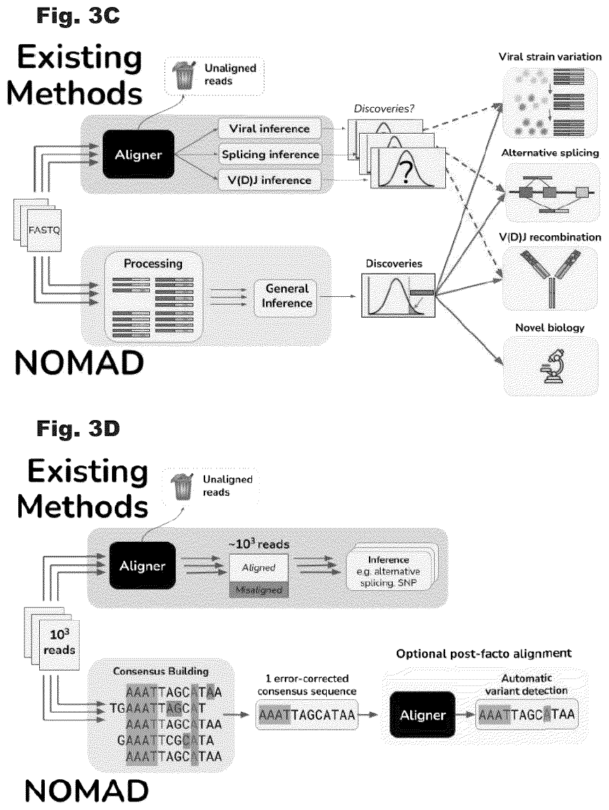

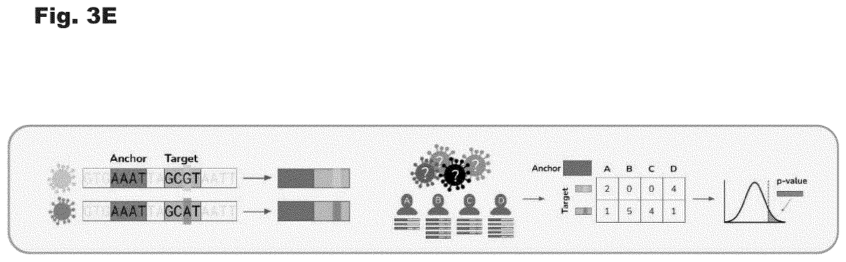

The systems and methods of the disclosure will be better understood with the several examples provided. Validation results are also provided. The examples show that detection of sample-dependent sequence diversification can be formulated probabilistically (see FIG. 3A). The examples further describe that the systems and methods for analyzing diverse nucleic acid samples are able to be reduced to a statistical test on raw sequencing read data (e.g. FASTQ files). The examples further provide data of exemplary systems and methods that are tested in a highly efficient algorithmic workflow, providing novel discovery across disparate biological disciplines (FIGS. 3B and 3C). Further details of examples can be found in the following publications: K. Chaung, et al., bioRxiv. 2022.06.24.497555 (2023) doi: 10.1101/2022.06.24.4975; R. Dehghannasiri, et al., bioRxiv. 2022.12.06.519414 doi: 10.1101/2022.12.06.519414; T. Baharav, et al., bioRxiv. 2023.03.16.533008; doi: 10.1101/2023.03.16.533008; and M. Kokot, et al., bioRxiv 2023.03.17.533189; doi: 10.1101/2023.03.17.533189; the disclosure of which are each incorporated herein by reference.

### Example 1: NOMAD is a Statistics-First Approach to Identify Sample-Dependent Sequence Diversification

To begin, an “anchor” k-mer can be defined in a read, and say the anchor has sample-dependent diversity if the distribution of k-mers starting R basepairs downstream of it (called “targets”) depends on the sample (FIG. 3E). Inference can be performed for much more general constructions of anchors and targets: any tuple of disjoint subsequences from DNA, RNA or protein sequence data can be analyzed in this framework.

This formulation unifies many fundamental problems in genome science. NOMAD (Novel multi-Omics Massive-scale Analysis and Discovery) is a novel statistics-first approach that is reference-free and operates directly on raw sequencing data. It is an extremely computationally efficient algorithm to detect sample-dependent sequence diversification, through the use of a novel statistic of independent interest that provides closed form p-values. NOMAD makes all predictions blind to references and annotations, though they can be optionally used for post-facto interpretation. This makes NOMAD fundamentally different from existing methods. To illustrate, as a special case NOMAD is detection of differential isoform expression. In this domain, the closest approach to NOMAD is Kallisto which requires a reference transcriptome and statistical resampling for inference, and is further challenged to provide exact quantification for more than a handful of paralogous genes and isoforms (for more on Kallisto, see N. L. Bray et al., Nat. Biotechnol. 34, 525-527 (2016), the disclosure of which is incorporated herein by reference). Unlike NOMAD, Kallisto cannot discover spliced isoforms de novo.

NOMAD's calls are “significant anchors”: sequences a where, given observing a in a read, the conditional distribution of observing a target sequence t a distance R downstream of a is sample-dependent (FIG. 3E). NOMAD is by default an unsupervised algorithm that does not require any sample identity metadata. It finds approximate best splits of data into two groups, or it can use user-defined groups if desired. Anchors are reported with an effect size in [0,1], a measure of target distribution difference between sample groups: 0 if the groups have no difference in target distributions and increasing to 1 when the target distributions of the two groups are disjoint. NOMAD has multiple major technical innovations: 1) a parallelizable, fully containerized, and computationally efficient approach to parse FASTQ files into contingency tables, 2) novel statistical analysis of the derived tables, using concentration inequalities to derive closed form p-values, 3) a micro-assembly-based consensus sequence representing the dominant error-corrected sequence, downstream of the anchor for post-facto interpretation and identification of SNPs, indels or isoforms, to name a few (FIG. 3D). If post anchor-identification inference is desired, the consensus, rather than raw reads, is aligned. This reduces the number of reads to align by ~1000× in real data.

Together, NOMAD's theoretical development yields an extremely computationally efficient implementation. NOMAD was run on a 2015 Intel laptop with an Intel® Core™ i7-6500U CPU @2.50 GHz processor, generating significance calls for single cell RNA-seq totaling over 43 million reads in only 1 hour 45 min. When performed on a computer cluster, the same analysis is completed in an average of 2.28 minutes with 750 MB of memory for 4 million reads, a dramatic speed up over existing methods for de novo splicing detection and significance calls.

### Anchor Processing

Following notation in J. Abante et al., anchors and targets are defined as contiguous subsequences of length k positioned at a distance R=max(0, (L−2*k)/2) apart (rounded), where L is the length of the first read processed in the dataset. If L=100 and k=27, then R=23 (J. Abante, P. L. Wang, and J. Salzman, BioRxiv (2022), doi:10.1101/2022.06.13.495703, the disclosure of which is incorporated herein by reference). Anchor sequences can be extracted as adjacent, disjoint sequences or as tiled sequences that begin at a fixed step size. In this example, NOMAD was run with 4M reads per FASTQ file, anchor sequences tiled by 5 bp, and k=27. To satisfy the independence assumption for computing p-values in the NOMAD statistics, only one read is used if the sequencing data is paired end; in this example, read 1 was used. Extracted anchor and target sequences are then counted for each sample with the UNIX command, ‘sort|uniq -c’, and anchor-target counts are then collected across all samples for restratification by the anchor sequence. This stratification step allows for user control over parallelization. To reduce the number of hypotheses tested and required to correct for, p-value calculation was used only for anchors with more than 50 total counts across all samples. Anchors were discarded that have only one unique target, anchors that appear in only 1 sample, and (anchor, sample) pairs that have fewer than 6 counts. An anchor was retained if anchor has more than 30 total counts after above thresholds were applied removals. This approach efficiently constructs sample by target counts matrices for each anchor. It was noted that for a fixed number of anchor-target pairs, under alternatives such as differential exon skipping, NOMAD analysis for larger choices of R have provably higher power than smaller choices.

NOMAD p-Values

While contingency tables have been widely analyzed in the statistics community, it is uncommon for tests to provide closed form, finite-sample valid statistical inference with desired power for the application at hand. A test statistic S was developed that has power to detect sample-dependent sequence diversity and is designed to have low power when there are a few outlying samples with low counts as follows. First, a function f was randomly constructed, which maps each target independently to {0,1}. The mean value of targets was then computed with respect to this function. Next, the mean within each sample of this function was computed. Then, the anchor-sample score was construct for sample j, Sj, as a scaled version of the difference between these two. Finally, the test statistic S was constructed as the weighted sum of these Sj, with weights cj (which denote class-identity in the two-group case with metadata and are chosen randomly without metadata, see below). In the below equations, Dj,k denotes the sequence of the k-th target observed for the j-th sample.

$$\hat{μ} = \frac{1}{M} {\sum }_{j , k} f ( {D}_{j , k} )$$

$${\hat{μ}}_{j} = \frac{1}{{n}_{j}} {\sum }_{k = 1}^{{n}_{j}} f ( {D}_{j , k} )$$

$${S}_{j} = \sqrt{{n}_{j}} ( {\hat{u}}_{j} - \hat{u} )$$

$$S = {\sum }_{j = 1}^{p} {c}_{j} {S}_{j}$$

This allows construction of statistically valid p-values as:

$$P = 2 exp ( - \frac{2 {( 1 - a )}^{2} {S}^{2}}{{\sum }_{j : {n}_{j} > 0} {c}_{j}^{2}} ) + 2 exp ( - \frac{2 {a}^{2} M {S}^{2}}{{( {\sum }_{j} {c}_{j} \sqrt{{n}_{j}} )}^{2}} ) with a = {( 1 + \sqrt{\frac{M {\sum }_{j} {c}_{j}^{2}}{{( {\sum }_{j} {c}_{j} \sqrt{{n}_{3}} )}^{2}}} )}^{- 1}$$

This statistic is computed for K different random choices of f, and in the case where sample group metadata is not available, jointly for each of the L random choices of c. The random choice of cj's was called “random c's” below. The choice of f and c that minimize the p-value are reported, and are used for computing the p-value of this anchor. To yield valid p-values Bonferroni correction was applied over the L*K multiple hypotheses tested (just K when sample metadata is used and randomization on c is not performed). Then, to determine the significant anchors, BY correction (BH with positive dependence) was applied to the list of p-values for each anchor, yielding valid FDR controlled q-values reported throughout the manuscript (Y. Benamini and D. Yekutieli Ann. Statist. 29, 1165-1188 (2001), the disclosure of which is incorporated herein by reference).

$${Q}_{( i )}^{BY} = min ( {min}_{j \geq i} \frac{m ( log m + 1 ) {p}_{( j )}}{j} , 1 )$$

### NOMAD Effect Size

NOMAD provides a measure of effect size when the cj's used are +/−1, to allow for prioritization of anchors with large inter-sample differences in target distributions. Effect size is calculated based on the split c and function f that yield the most significant NOMAD p-value. Fixing these, the effect size is the absolute value of the difference between the mean function value over targets (with respect to f) across those samples with cj=+1 denoted A+, and the mean over targets (with respect to f) across those samples with cj=−1 denoted A−.

$$\vert \frac{1}{{\sum }_{j ∈ {A}_{+}} {n}_{j}} {\sum }_{j ∈ {A}_{+}} {n}_{j} {\hat{u}}_{j} - \frac{1}{{\sum }_{j ∈ {A}_{-}} {n}_{j}} {\sum }_{j ∈ {A}_{-}} {n}_{j} {\hat{u}}_{j} \vert$$

This effect size has natural relations to a simple 2 group alternative hypothesis. It can also be shown to relate to the total variation distance between the empirical target distributions of the two groups.

### Consensus Sequences

A consensus sequence is built for each significant anchor for the sequence downstream of the anchor sample. A separate consensus is built for each sample by aggregating all reads from this sample that contain the given anchor. Then, NOMAD constructs the consensus as the plurality vote of all these reads; concretely, the consensus at basepair i is the plurality vote of all reads that contain the anchor, i basepairs after the anchor appears in the read (a read does not vote for consensus base i if it has terminated within i basepairs after the anchor appeared). The consensus base as well as the fraction agreement with this base among the reads is recorded.

The consensus sequences can be used for both splice site discovery and other applications, such as identifying point mutations and highly diversifying sequences, e.g. V(D)J rearrangements. The statistical properties of consensus building make it an appealing candidate for use in short read sequencing, and may have information theoretic justification in de novo assembly.

To provide intuition regarding the error correcting capabilities of the consensus, consider a simple probabilistic model where reads from a sample all come from the same underlying sequence. In this case, under the substitution only error model, the probability that the consensus for n reads makes a mistake at a given location i under independent sequencing error rate epsilon (substitution only) is at most

$$ℙ ( error at basepair i ) \leq {\sum }_{k \geq n / 2}^{n} ( \begin{matrix}n \\ k\end{matrix} ) {ϵ}^{k} {( 1 - ϵ )}^{n - k} \geq \frac{n}{2} ( \begin{matrix}n \\ n / 2\end{matrix} ) {ϵ}^{n / 2}$$

It can be seen that even for n=10, this probability is less than 1.3E-7 for a given basepair, which can be union-bounded over the length of the consensus to yield a vanishingly small probability of error. Thus, for a properly aligned read, if a basepair differs between the consensus and reference it is almost certainly a SNP.

### Element Annotations

To identify false positive sequences or contextualize mobile genetic elements, anchors and targets are aligned with bowtie2 to a set of indices, corresponding to databases of sequencing artifacts, transposable elements, and mobile genetic elements. In these alignments, using bowtie2, the best hit is reported, relative to an order of priority (13). The reference used, in order of priority, are: UniVec, Illumina adapters, Escherichia phage phiX174, Rfam (45), Dfam (46), TnCentral (47), ACLAME (48), ICEberg (49), CRISPR direct repeats (50), ITSoneDB (51), ITS2 (52), WBcel235, TAIR10, grch38_1kgmaj (for more on references, see 1. Kalvari, et al., Nucleic Acids Res. 49, D192-D200 (2021); J. Storer, et al., Mob. DNA. 12, 2 (2021); K. Ross, et al., MBio. 12, e0206021 (2021); R. Leplae, et al., Nucleic Acids Res. 32, D45-9 (2004); D. Bi, et al., Nucleic Acids Res. 40, D621-6 (2012); D. Couvin, et al., Nucleic Acids Res. 46, W246-W251 (2018); M. Santamaria, et al., Nucleic Acids Res. 46, D127-D132 (2018); and C. Selig, et al., Nucleic Acids Res. 36, D377-80 (2008); the disclosures of which are each incorporated herein by reference). To perform these annotations, bowtie2 indices were built from the respective reference fastas, using bowtie2-build with default parameters. Anchors and targets were then aligned to each index, using bowtie2-align with default parameters. For each sequence, the alignment to the reference and the position of that alignment for each reference in the prespecified set was reported. Anchors and targets, and their respective element annotations, are reported in the element annotation summary files.

### Genome Annotations

Anchor, target, and consensus sequences can be aligned to reference genomes and transcriptomes, to provide information about the location of sequences relative to genomic elements.

For significant anchors, target, and consensus sequences, information regarding the anchor, target, and consensus sequences' alignments to both a reference genome and transcriptome can be reported in a genome annotation summary file. All alignments reported below are run in two modes in parallel: bowtie2 end-to-end mode (the bowtie2 default parameters) and bowtie2 local mode (‘-local’, in addition to the bowtie2 default parameters); the following columns are prefixed with “end_to_end” or “local”, for end-to-end mode and local mode, respectively.

To report alignments to the transcriptome, the sequences are aligned to the reference transcriptome with bowtie2, with ‘-k 1’, in addition to the above parameters, to report a maximum of one alignment per sequence. If there is a transcriptome alignment, the alignment to the reference and the MAPQ score of the alignment can be reported.

To report alignments to the genome, the sequences are aligned to the reference genome, with the same parameters above. If there is a genome alignment, alignment to the reference, the strand of the alignment, and the alignment MAPQ score can be reported. To lend further context, any annotated gene intersection to the reference genome alignment can be reported by first converting the genome alignments to BED format and then using ‘bedtools intersect’ on the genome alignments BED file and a BED file of gene annotations (for example, hg38 RefSeq can be used); for each sequence, the list of distinct gene intersections per sequence genome alignment can be reported. For each sequence genome alignment, its distances to the nearest annotated exon junctions, by using ‘bedtools closest’ with the sequence genome alignments and BED files of annotated exon start and end coordinates can be reported. To report distances of a sequence genome alignment to the nearest upstream exon starts and nearest upstream exon starts, the closest exon start and exon end can be reported, respectively, with the ‘bedtools closest’ parameters ‘-D ref -id -t first’. To report distances of a sequence genome alignment to the nearest downstream exon starts and nearest downstream exon starts, the closest exon start and exon end can be reported, respectively, with the ‘bedtools closest’ parameters ‘-D ref -iu -t first’.

### NOMAD Protein Profiles

For each set of enriched anchors, homology-based annotation was attempted against an annotated protein database, the Pfam (J. Mistry, et al., Nucleic Acids Res. 49, D412-D419 (2021), the disclosure of which is incorporated herein by reference). For each dataset, up to 1000 of the most significant anchors (q-value<0.01) were retained for the following analysis: a substring of each downstream consensus was generated by appending each consensus nucleotide assuming both conditions were met: a minimum observation count of 10 and a minimum agreement fraction of 0.8, until whichever metric first exhibited two consecutive failures at which point no further nucleotide was added. A limit of 1000 anchors was used due to computational constraints from HMMer3 (see below). Anchors that did not have any consensus nucleotides appended were kept as is. An extended anchor was generated for each experiment in which an anchor was found. Each extended anchor was then stored in a final concatenated multi FASTA file with unique seqID headers for each experiment's extended anchors.

The number of matched anchors used for NOMAD and control analysis per dataset are as follows: 201 high effect size anchors in SARS-CoV-2 from South Africa, 252 high effect size anchors in SARS-CoV-2 from France; 1000 anchors were used for rotavirus, human T cells, human B cells, Microcebus natural killer T cells, and Microcebus B cells.

To assess these extended anchors for protein homology, this concatenated FASTA file was then translated in all six frames using the standard translation table using seqkit prior to using hmmsearch from the HMMer3 package to assess each resulting amino acid sequences against the Pfam35 profile Hidden Markov Model (pHMM) database (see W. Shen, et al., PLoS ONE. 11, e0163962 (2016); and L. S. Johnson, et al., BMC Bioinformatics. 11, 431 (2010); the disclosures of which are each incorporated herein by reference.

All hits to the Pfam database were then binned at different E-value orders of magnitude and plotted. In each case, control assessments were performed by repeating the extension and homology searches against an equivalent number of control anchors, selected as the most frequent anchors from that dataset.

Lastly it is worth noting that while only counts of the best scoring Pfam hits were assessed in this study, other information is also produced by HMMer3. In particular, relative alignment positions are given for each hit which could be used to more finely pinpoint the precise locus at which sequence diversification is detected.

We note that while the number of input anchors for NOMAD and control sets are matched, it is possible to have more control protein domains in the resulting barplots, as only high E-value hits to Pfam are reported in the visualizations.

### Control Analysis

To construct control anchor lists based on abundance, all anchors input to NOMAD were considered and their abundance was counted, collapsing counts across targets. That is, an anchor receives a count determined by the number of times it appears at an offset of 5 in the read up to position R− max(0,R/2-2*k) where R is the length of the read, summed over all targets. The 1000 most abundant anchors were output as the control set. For analysis comparing control to NOMAD anchors, min(|NOMAD anchor list|,1000) most abundant anchors from the control set were used and the same number of NOMAD anchors were used, sorted by p-value.

### Example 2: NOMAD Discovers Sequence Diversification in Proteins at the Host-Viral Interface without a Genomic Reference

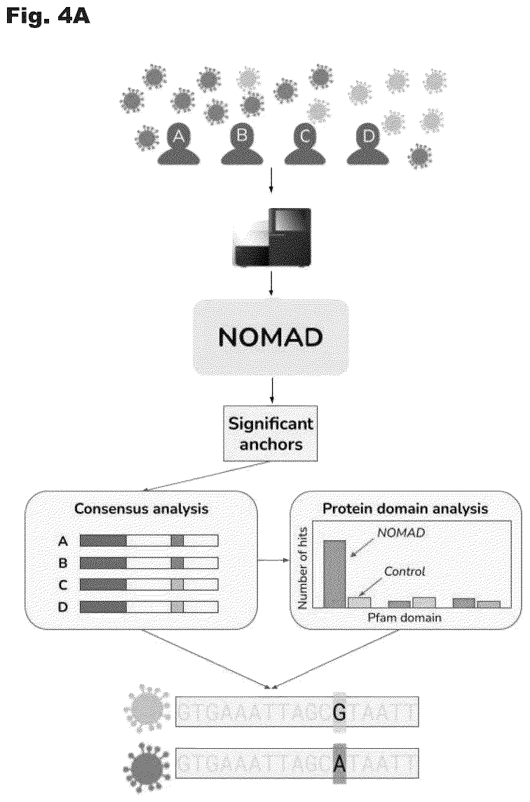

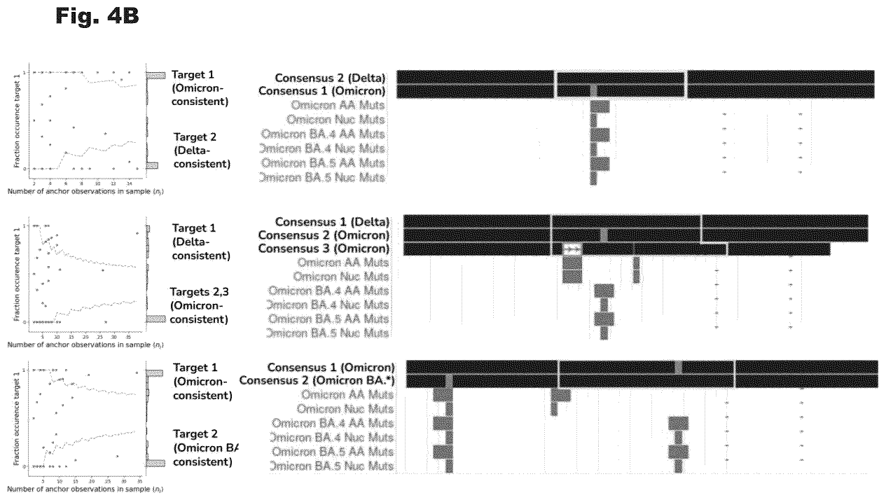

NOMAD can automatically detect viral strain evolution without any knowledge of the sample origin. Existing approaches are computationally-intensive, require genome assemblies and rely on heuristics. Yet, emergent viral threats or variants of concern, e.g. within SARS-CoV-2, will necessarily be absent from reference databases. Because virus' genomes are under selective pressure to diversify when infecting a host, NOMAD can prioritize anchors near genome sequences that are under selection, in theory, based purely on their statistical features: sequences flanking variants that distinguish strains have consistent sample-dependent sequence diversity. When NOMAD is run on patients differentially infected by Omicron and Delta strains of SARS-CoV-2, significant anchors are expected to be called adjacent to strain-specific mutations (FIG. 4A); they are discoverable without any knowledge of a reference.

NOMAD's ability to discover strain-specific mutations, Oropharyngeal swabs from patients with SARS-CoV-2 from 2021 Dec. 6 to 2022 Feb. 27 in France, a period of known Omicron-Delta coinfection were analyzed (A. Bal, et al., medRxiv (2022), doi:10.1101/2022.03.24.22272871, the disclosure of which is incorporated herein by reference). NOMAD analyzed anchors with effect size>0.5, as high effects are predicted if samples can be approximately partitioned by strain, though results are similar without this threshold. For each anchor, a protein domain label was assigned based on in silico translation of its consensus sequence. The protein domain with best mapping to the Pfam database is assigned to the anchor, producing a set of “NOMAD protein profiles” and is compared to matched controls. NOMAD protein profiles are significantly different from controls (p=1.1E-12, chi-squared test). The most NOMAD-enriched domains are the receptor binding domain of the betacoronavirus spike glycoprotein (7 NOMAD vs 0 control hits, p=2.9E-4 hypergeometric p-value, corrected,) and Orf7A a transmembrane protein that inhibits host antiviral response (6 NOMAD vs 0 control hits, p=1.6E-3 hypergeometric p-value, corrected). All analysis is blind to the data origin (SARS-CoV-2).

Patient samples from the original South African genomic surveillance study that identified the Omicron strain during the period 2021 Nov. 14 to 2021 Nov. 23 were also analyzed, again without metadata, reference genomes and directly on input FASTQ files (R. Viana, et al., Nature. 603, 679-686 (2022), the disclosure of which is incorporated herein by reference). NOMAD protein profiles were significantly different from controls (chi-squared test, p=2.5E-39). NOMAD-enriched domains in France and South African are highly consistent: the top four domains in both datasets are permutations of each other. The most NOMAD-enriched domains versus controls were the betacoronavirus S2 subunit of the spike protein involved in eliciting the human antibody response (23 NOMAD vs 2 control hits, p=2.9E-6 hypergeometric p-value, corrected), the matrix glycoprotein which interacts with the spike (20 NOMAD vs 0 control hits, p=8.4E-8 hypergeometric p-value, corrected), and the receptor binding domain of the spike protein and to which human antibodies have been detected (20 NOMAD vs 0 control hits, p=8.4E-8 hypergeometric p-value, corrected) (see P. Jörrißen, et al., Front. Immunol. 12, 679841 (2021), the disclosure of which is incorporated herein by reference). All domains are biologically predicted to be under strong selective pressure; NOMAD discovers this de novo.

NOMAD anchors with effect size>0.5 to were aligned the Wuhan reference strain to test if NOMAD anchors rediscovered known strain-defining mutations. A mutation-consistent anchor was defined as one consistent with detecting an Omicron or Delta variant mutation. NOMAD anchors are significantly enriched for being mutation-consistent: in the French data, of the uniquely mapped anchors (Wuhan reference), 30.5% (44/144) of NOMAD's calls were mutation consistent versus 7.6% (6/78) in the control (p=4.0E-5, hypergeometric test). For the South African data, 67.9% (89/131) of NOMAD's called anchors were mutation consistent vs 20% (12/60) for the control (p=4.4E-10, hypergeometric test).

Examples of mutation-consistent anchors are presented in FIG. 4B including in the membrane (FIG. 4B, top) and spike protein (FIG. 4B, middle and bottom). Differences between consensus sequences and Wuhan reference illustrate NOMAD's unsupervised rediscovery of annotated strain-specific variants, including co-detection of a deletion and a mutation (FIG. 4B). While the Wuhan reference genome was used for post-facto interpretation, no alignment or sample metadata was used to generate NOMAD's calls, only to interpret them (FIG. 2). NOMAD consensuses also extend discovery (FIG. 4B), identifying strain-specific variants beyond the target: both are annotated omicron variants (FIG. 4B). NOMAD's statistical approach automatically links discovered variants within patients de novo: one consensus contains the omicron Variant of Concern (VOC) T22882G; a second consensus has a single VOC T22917G identified in Omicron strains BA.4 and BA.5 in May of 2022, 3 months after the analyzed samples were collected; a third consensus contains the VOC as well as the VOC G22898A; a single further consensus shows no mutations, consistent with Delta infection. Together, this suggests that mutations in BA.4 and BA.5 were circulating well before the VOC was called in May 2022.

499 samples collected in California (2020) before viral strain divergence in the spike had been reported were analyzed as a negative control (see J. E. Gorzynski, et al., medRxiv (2020), doi:10.1101/2020.07.27.20163147, the disclosure of which is incorporated herein by reference). No enrichment of NOMAD protein profiles related to the spike or Orf7a domains were observed, supporting the idea that NOMAD calls are not false positives. To explore the generality of NOMAD for reference-free discovery in other viral infections, NOMAD was additionally run on a study of influenza-A and of rotavirus breakthrough cases. NOMAD Protein profile analysis showed enrichment in domains involved in viral suppression of the host response and regulated alternative splicing. Together, this suggests that NOMAD analysis could aid in viral surveillance, including detecting emergence of variants of concern directly from short read sequencing, bypassing a requirement for reference genomes and without manual scrutiny of individual samples or their assembled genomes.

### SARS-CoV2 Analysis

The SARS-CoV2 datasets used in this example were analyzed with NOMAD's unsupervised mode (no sample metadata provided). To identify high effect size anchors, a threshold of ‘effect_size_randCjs’>0.5 was used (table S2). The Wuhan variant reference genome was downloaded from NCBI, assembly NC_045512.2. The Omicron and Delta mutation variants were downloaded as FASTA from the UCSC track browser in June 2022, with the following parameters: clade ‘Viruses’, assembly NC_045512.2, genome ‘SARS-CoV-2′, group ‘Variations and Repeats’, track ‘Variants of Concert’, and table ‘Omicron Nuc Muts (variantNucMuts_B_1_1_529)’ and’Delta Nuc Muts (variantNucMutsV2_B_1_617_2)’.

Variant genomes were downloaded in FASTA file format, and bowtie indices were built from these FASTA files, using default parameters. To determine alignment of anchors to the Wuhan genome, anchor sequences were converted to FASTA format and aligned to the Wuhan bowtie index with bowtie (default parameters). After mapping of NOMAD anchors, the number of control anchors were chosen to match the number of anchors mapped by bowtie to report comparable numbers.

Mutation consistency to the Omicron and Delta variants was reported as follows. For each anchor mapping to the Wuhan reference in the positive strand, an anchor at position x is called mutation-consistent if there is an annotated variant between positions x+k+D and x+R+2*D, where D=max(0, (L−2*k)/2), L is the length of the first read processed in the dataset, and the factor of 2 reflects the bowtie convention of reporting the left-most base in the alignment. The reciprocal logic was used to define mutation-consistency for anchors mapping to the negative strand—e.g. a mutation had to occur between positions x−(R+D) and x. In total, the report included: a) number of anchors mapping with bowtie default parameters to the Wuhan reference; b) number and fraction of mutation-consistent anchors as described above.

### Viral Protein Profile Analysis

In influenza, NOMAD's most frequently hit profiles were Actin (62 hits), and GTP_EFTU (23 hits), and the influenza-derived Hemagglutinin (17 hits), consistent with virus-induced alternative splicing of Actin and EF-Tu, further elucidating these proteins' roles during infection (no such hits were found in the control). Similarly, in a study of metagenomics of rotavirus breakthrough cases, NOMAD protein profile analysis prioritized domains known to be involved in host immune suppression.

In rotavirus, the most enriched domain in NOMAD compared to control was the rotavirus VP3 (Rotavirus_VP3, 76 NOMAD hits vs 9 control hits), a viral protein known to be involved in host immune suppression, and the rotavirus NSP3 (Rota_NSP3, 87 NOMAD vs 35 control hits), a viral protein involved in subverting the host translation machinery, both proteins that might be expected to be under constant selection given their intimate host interaction.

### Example 3: NOMAD Discovers Isoform-Specific Expression in Single Cell RNA-Seq

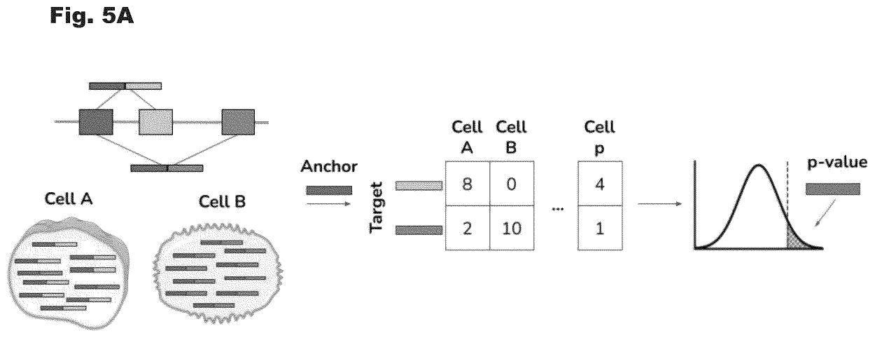

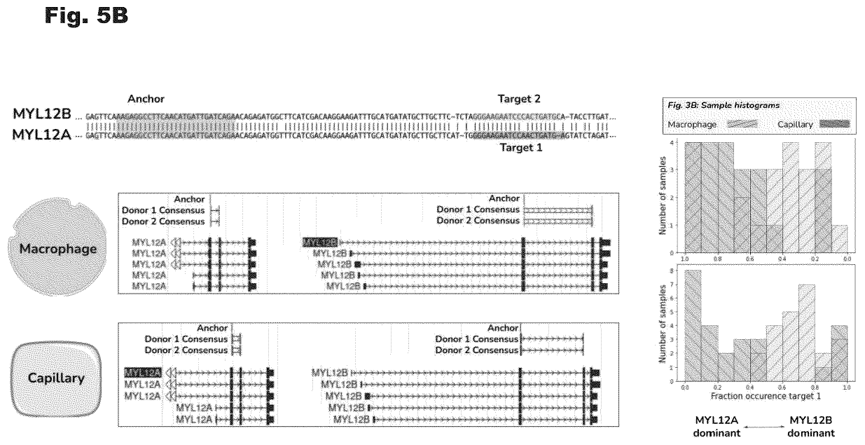

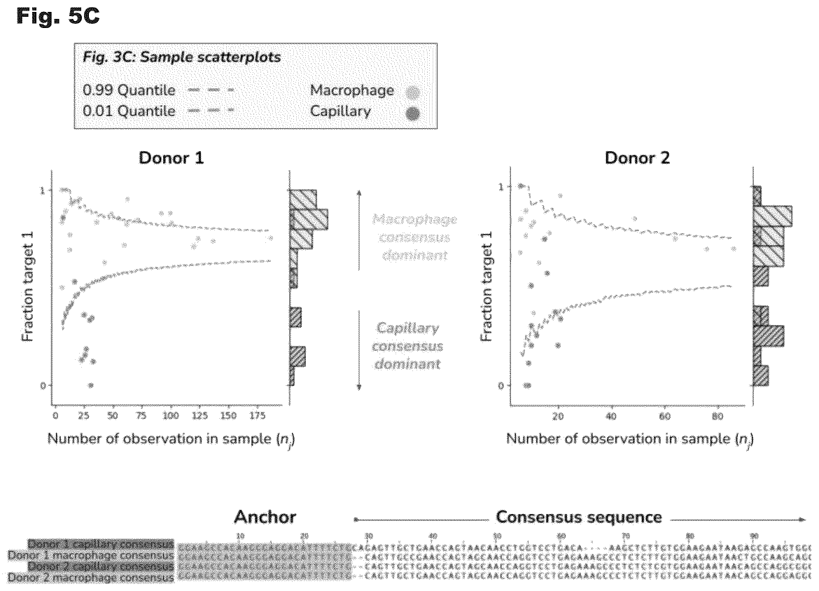

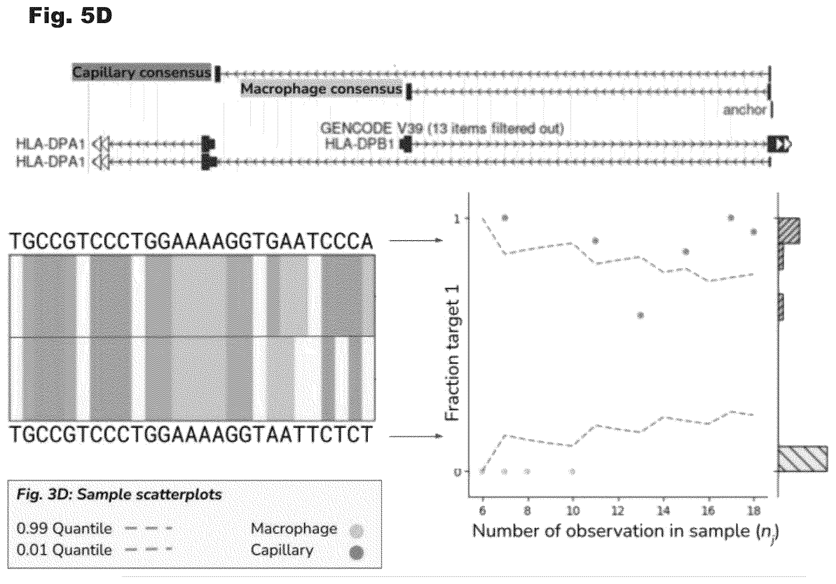

NOMAD is a general algorithm to discover sample-dependent sequence diversification in disparate applications including RNA expression and beyond. To illustrate the former, NOMAD was run without any parameter tuning on single cell RNA-seq datasets, testing if it could perform the fundamental but previously distinct tasks of identifying regulated expression of paralogous genes, alternative splicing and V(D)J recombination (FIG. 5A).

First, NOMAD was tested to see if it discovers alternatively spliced genes in single cell RNA-seq (Smart-seq 2) of human macrophage versus capillary lung cells, chosen because they have a recently established positive control of alternative splicing, MYL6, a subunit of the myosin light chain (see, J. E. Olivieri, et al., eLife. 10 (2021), doi:10.7554/eLife.70692, the disclosure of which is incorporated herein by reference). NOMAD rediscovered MYL6 and made new discoveries not reported in the literature. For example, NOMAD discovered reproducible cell-type specific regulation of MYL12 isoforms, MYL12A and MYL12B. Like MYL6, MYL12 is a subunit of the myosin light chain. In humans (as in many species) two paralogous genes, MYL12A and MYL12B, sharing >95% nucleotide identity in the coding region, are located in tandem on chromosome 18 (FIG. 5B).

Reference-first algorithms fail to quantify differential expression of MYL12A and MYL12B due to mapping ambiguity. NOMAD automatically detects targets that unambiguously distinguish the two paralogous isoforms, and demonstrates their clear differential regulation in capillary cells and macrophages (FIG. 5B). MYL12 isoform specificity was confirmed in pairwise comparisons of the same cell types in two further independent single cell sequencing studies of primary cells from the same cell-types. MYL12 was recently discovered to mediate allergic inflammation by interacting with CD69; while today little is known about differential functions of the two MYL12 paralogs, the distinct roles of highly similar actin paralogs provides a precedent.

NOMAD also called reproducible cell-type specific allelic expression and splicing in the major histocompatibility (MHC) locus (FIG. 5C), the most polymorphic region of the human genome which carries many significant disease risk associations. Despite its central importance in human immunity and complex disease, allotypes are difficult to quantify, and statistical methods to reliably distinguish them do not exist. NOMAD finds (i) allele-specific expression of HLA-DRB within cell types and (ii) cell-type specific splicing, predicted to change the amino acid and 3′ UTR sequence of HLA-DPA1 (FIG. 5D). These empirical results bear out a snapshot of the theoretical prediction that NOMAD's design gives it high statistical power to simultaneously identify isoform expression variation and allelic expression, including that missed by existing algorithms.

### Identifying Cell-Type Specific Isoforms in SS2

In the analysis of HLCA SS2 data, “isoform detection conditions” was utilized for alternative isoform detection. These conditions select for (anchor, target) pairs that map exclusively to the human genome, anchors with at least one split-mapping consensus sequence, mu_lev>5, and M>100. mu_lev is defined as the average target distance from the most abundant target as measured by Levenstein distance. To identify anchors and targets that map exclusively to the human genome, anchors and targets that had exactly one element annotation were included, where that one element annotation must be grch38_1 kgmaj. To identify anchors with at least one split-mapping consensus, anchors that had at least one consensus sequence with at least 2 called exons were selected. The conditions on Levenshtein distance, designed to require significant across-target sequence diversity, significantly reduced anchors analyzed (excluding many SNP-like effects). The set of anchors was further restricted to anchors with M>100 to account for the lower cell numbers in macrophage cells; note that the user can perform inference with a lower M requirement, based on input data. These isoform detection parameters were used to identify the SS2 examples discussed in this example, MYL12. For HLA discussion, gene names were called using consensus_gene_mode.

### Splice Junction Calls

To identify exon coordinates for reporting annotations in this example, consensus sequences are mapped with STAR aligner (default settings) (A. Dobin, et al., Bioinformatics. 29, 15-21 (2013), the disclosure of which is incorporated herein by reference). Gapped alignments are extracted and their coordinates are annotated with known splice junction coordinates using ‘bedtools bamtobed --split’; each resulting contiguously mapping segment is called a “called exon” (see below). From each consensus sequence, called exons are generated as start and end sites of each contiguously mapped sequence in the spliced alignment. These ‘called exons’ are then stratified as start sites and end sites. Note that the extremal positions of all called exons would not be expected to coincide with a splice boundary (see below); “called exon” boundaries would coincide with an exon boundary if they are completely internal to the set of called exon coordinates. Each start and end site of each called exon is intersected with an annotation file of known exon coordinates; it receives a value of 0 if the site is annotated, and 1 if it is annotated as alternative. The original consensus sequence and the reported alignment of the consensus sequence are also reported. Gene names for each consensus are assigned by bedtools intersect with gene annotations (hg38 RefSeq for human data by default), possibly resulting in multiple gene names per consensus.

### Example 4: Unsupervised Discovery of B, T Cell Receptor Diversity from Single-Cell RNA-seq

NOMAD was assessed to determine if it could identify T cell receptor (TCR) and B cell receptor (BCR) variants. B and T cell receptors are generated through V(D)J recombination and somatic hypermutation, yielding sequences that are absent from any reference genome and cannot be cataloged comprehensively due to their diversity (>1012). Existing methods to identify V(D)J rearrangement require specialized workflows that depend on receptor annotations and alignment and thus fail when the loci are unannotated. In many organisms, including Microcebus murinus, the mouse lemur, T cell receptor loci are incompletely unannotated as they must be manually curated.

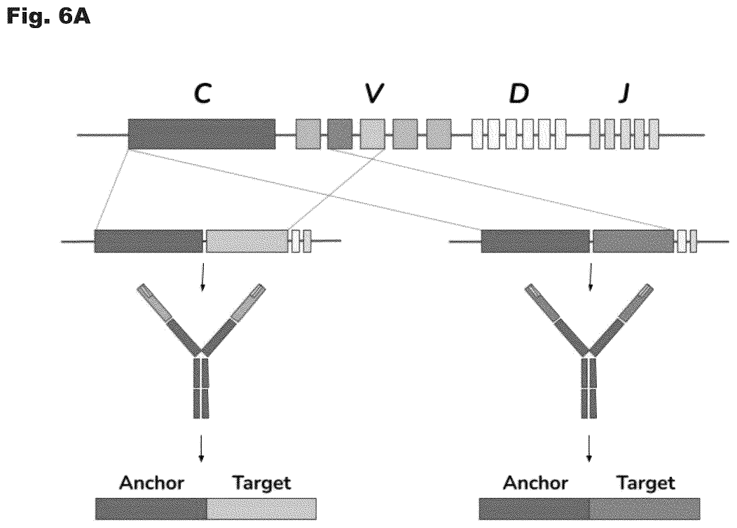

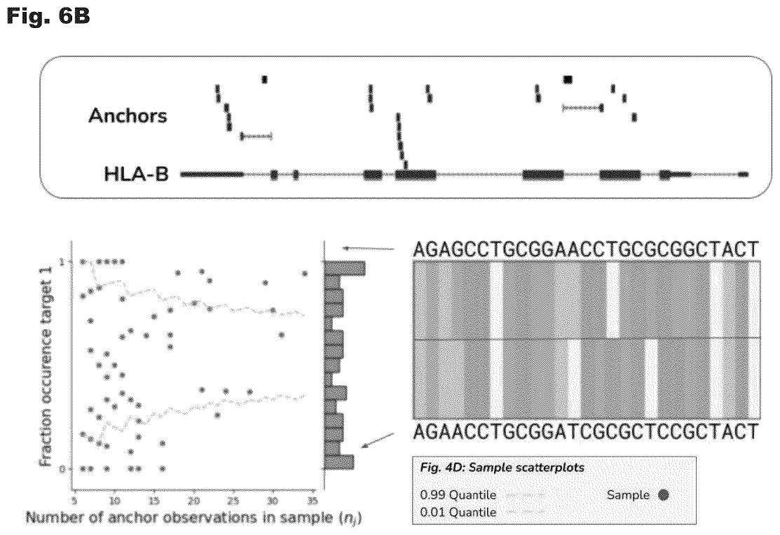

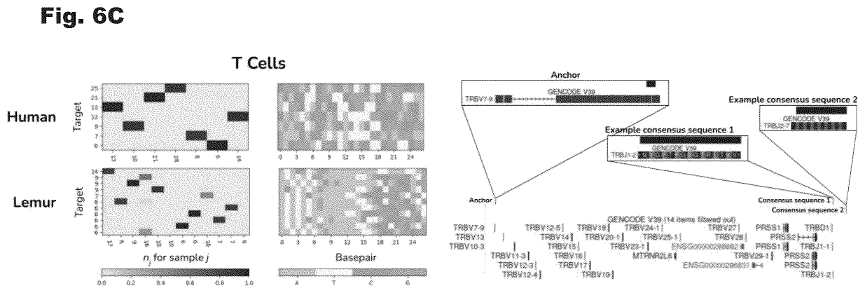

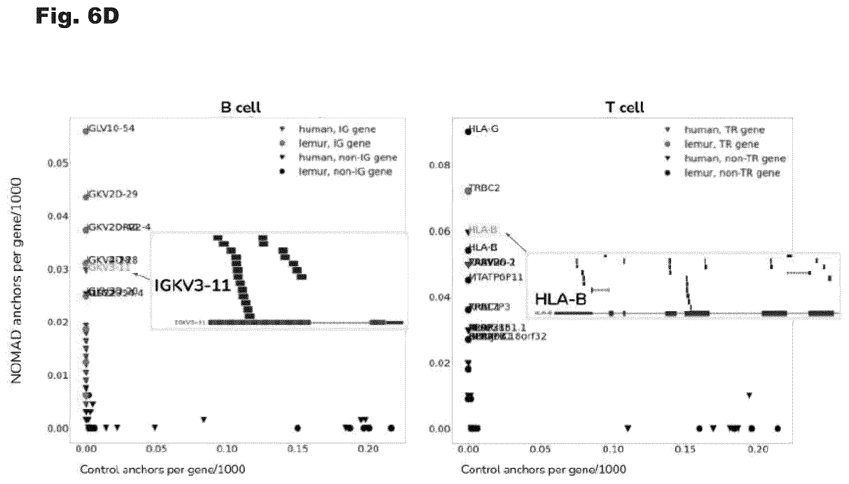

NOMAD was tested if it could identify TCR and BCR rearrangements in the absence of annotations on 111 natural killer T and 289 B cells isolated from the spleen of two mouse lemurs (Microcebus murinus) profiled by Smart-Seq2 (SS2), and performed the same analysis on a random choice of 50 naive B cells from the peripheral blood and 128 CD4+ T cells from two human donors profiled with SS2 for comparison (FIG. 6A).

NOMAD protein profiles, which are blind to the sample's biological origin and do not require any reference genome, revealed that NOMAD's most frequent hits in lemur B cell were IG-like domains resembling the antibody variable domain (86 hits), and COX2 (55 hits) a subunit of cytochrome c oxidase, known to be activated in the inflammatory response.

NOMAD's top hits for Lemur T cell were COX2 and MHC_I (77 and 58 hits, respectively). Similar results were obtained for the human samples. They include novel predictions of cell-type specific allelic expression of HLA-B in T cells (FIG. 6B) where NOMAD found statistical evidence that cells preferentially express a single allele (p<4,6E-24); consensus analysis shows SNPs in HLA-B are concordant with known positions of polymorphism.

NOMAD further predicted that BCR and TCR rearrangements would also be discovered by investigating the transcripts most hit by NOMAD anchors. NOMAD-called lemur B and T cell anchors were mapped to an approximation of its transcriptome: that from humans which diverged from lemur ~60-75 million years ago. Lemur B cell anchors most frequently hit the immunoglobulin light and kappa variable regions; lemur T cell anchors most frequently hit the HLA and T cell receptor family genes (FIG. 6C). Similar results are found in human B and T cells (FIG. 6D). Transcripts with the most hits in the control were unrelated to immune function. To further illustrate NOMAD's power, consider its comparison to existing pipelines. They cannot be run without the annotation for the lemur TCR locus; for assembling BCR sequences, pipelines e.g. BASIC cannot always identify V(D)J rearrangement, including in some cells profiled in the lemur dataset (see, S. Canzar, et al., Bioinformatics. 33, 425-427 (2017); and The Tabula Microcebus Consortium, et al., BioRxiv (2021), doi:10.1101/2021.12.12.469460; the disclosures of which are incorporated herein by reference). The 35 B/plasma cells where BASIC could not programmatically identify variable gene families on the light chain variable region were selected. NOMAD automatically identified anchors mapping to the IGLV locus, with consensus sequences that BLAST to the light chain variable region. Together, this shows that NOMAD identifies sequences with adaptive immune function including V(D)J in both B and T cells de novo, using either no reference genome (protein profile analysis) or only an annotation guidepost from a related organism (human). In addition to being simple and unifying, NOMAD can extend discovery compared to custom pipelines.

### HLA Analysis in HLCA

NOMAD summary files were processed by restricting to anchors aligning to the human genome, and having at least 1 target with this characteristic. Further, mu_lev had to exceed 1.5. For HLA discussion, gene names were called using consensus_gene_mode.

### B,T Cell Transcriptome Annotations

To determine the most frequent transcriptome annotation for a dataset, all significant anchors were mapped to the human transcriptome (GRCh38, Gencode) with bowtie2, using default parameters and ‘-k 1’ to report at most one alignment per anchor. Then, the bowtie2 transcript hits are aggregated by counting over anchors. The transcript hits with the highest counts over all anchors were reported.

### Further Immune Cell Protein Domain Analysis

In human B and T cells, NOMAD blindly rediscovered the high degree of single-cell variability in the immunoglobulin (IG) in B cells: this locus was most highly ranked by anchor counts per transcript (FIG. 6D). In B cells, NOMAD anchor counts were highest in genes IGKV3-11, IGKV3D-20, IGKV3D-11, and IGKC, the first three being variable regions of the B cell receptor (FIG. 6D).

Parallel analysis of T cells showed similar rediscovery and extension of known biology: HLA-B, RAP1B, TRAV26-2, and TRBV20-1 were the highest-ranked transcripts in T cells measured by anchor counts. HLA-B is a major histocompatibility (MHC) class I receptor known to be expressed in T cells, and TRAV26-2 and TRBV20-1 are variable regions of the T cell receptor. T cell expression of HLA-B alleles has been correlated with T cell response to HIV. FIG. 6D shows many other genes known to be rearranged by V(D)J were also recovered. In the control sets for both B and T cells, enriched genes were unrelated to immune functions.

HLA-B is the most densely hit transcript in T cells (FIG. 6D). Mapping assembled consensuses shows two dominant alleles: one perfectly matches a reference allele, the other has 4 polymorphisms all corresponding with positions of known SNPs. NOMAD statistically identifies T cell variation in the expression of these two alleles, some T cells having only detectable expression of one but not the other (p<4,6E-24). Other HLA alleles called by NOMAD, including HLA-F, have similar patterns of variation in allele-specific expression (Supplement).

### NOMAD Comparison to BASIC Analysis in Lemur Spleen B Cells

To compare performance, BASIC was run on the lemur spleen B cells, with the following additional parameters: -a. NOMAD was run on cells where BASIC failed to identify the light chain variable gene family, by selecting cells annotated as “No BCR light chain” from the BASIC output. From the NOMAD output, anchors which mapped to the IGL gene by bowtie were identified; to do this, the command ‘grep IGL “$file”’ was used, where “$file” corresponds to the NOMAD anchor genome annotations output file. This resulted in the following 5 anchors: CCTCAGAGGAGGGCGGGAACAGCGTGA, CTCGGTCACTCTGTTCCCGCCCTCCTC, GCCCCCTCGGTCACTCTGTTCCCGCCC, GGGCGGGAACAGCGTGACCGAGGGGGC, TCACTCTGTTCCCGCCCTCCTCTGAGG. SEQ ID NOs: 1-5.

The consensus sequences associated with the above IGL-mapping anchors were collected and converted into FASTA format. The following command was run on that FASTA file (denoted by “$fasta”): ‘blastn -outfmt “$fmt”-query “$fasta”-remote -db nt -evalue 0.1 -task blastn -dust no -word_size 24 -reward 1 -penalty -3-max_target_seqs 200’, where $fmt corresponds to “6 qseqid sseqid pident length mismatch gapopen qstart qend sstart send evalue bitscore sseqid sgi sacc slen staxids stitle”.

From this BLAST output, light chain variable regions were identified, via grep for the term “light chain variable”, yielding 60 sequences. Each cell could have at most 5 contributions to this number, and thus at least 12 cells (conservatively) had NOMAD-identified partial light chain variable sequences.

### Example 5: NOMAD+: An Integrated, Reference Free Pipeline to Discover Regulated RNA Expression

NOMAD's core includes a novel statistical test to detect sample-specific sequence variation that fills a gap in existing methods. Classical and parametric tests struggle to prioritize biologically important variation because they are overpowered in the context of noise generated from biochemical sampling, and such approaches may report inaccurate p-values. NOMAD's test provides finite-sample valid p-value bounds and, unlike Pearson's chi-squared test, controls false positive calls under commonly used modeling regimes such as negative binomial for scRNA-seq (see T. Baharav, et al., bioRxiv 2023.03.16.533008; doi: doi.org/10.1101/2023.03.16.533008; the disclosure of which is incorporated herein by reference). NOMAD's test performs inference in scRNA-seq independent of any cell metadata (e.g., cell type), which can be difficult to generate and remains imprecise and can miss important variation within cell types, such as B cell receptor variation.

In this example, NOMAD+ builds upon NOMAD to provide new approaches to analyze the output, including a new, simple reference-free statistical approach to de novo assembly as well as a framework to interpret its results (FIGS. 7A and 7B). NOMAD+ was used to discover extensive RNA transcript diversification in 10,326 human single cells profiled using SmartSeq2 from 136 cell types and 12 donors from the Tabula Sapiens project (Tabula Sapiens Consortium, et al., Science 376 (6594): eabl4896 (2022), the disclosure of which is incorporated herein by reference). NOMAD+ reveals new insights into the biology of single-cell regulation of transcript diversification—including features of RNA splicing, editing, and non-coding RNA expression missed by specialized, domain-specific bioinformatic pipelines. NOMAD+ detects sequences that have no known mapping to T2T, the latest human genome assembly (N. Altemose, et al., Science 376 (6588): eabl4178 (2022), the disclosure of which is incorporated herein by reference). Novel findings include (i) regulated expression of repetitive loci: RNU6 variants and of higher order repeats in centromeres including significant variation missed by mapping to the T2T reference genome; (ii) complex splicing programs including un-annotated variants in genes such as CD47, a major cancer immunotherapy target; (iii) pan-tissue regulation of splicing in splicing factors, histone regulation, and in the heat shock protein HSP90AA1; (iv) de novo rediscovery of immunoglobulin loci as the most transcriptionally diverse human loci with improved sensitivity; and (v) single cells with transcribed repeat expansion and high levels of RNA editing. NOMAD+ makes discoveries that are impossible with existing algorithms, without cell metadata or reference genomes, avoiding biases towards alignments to genomes best curated for European ancestries. The results herein suggest NOMAD+ is a single algorithm that could replace the myriad custom bioinformatic approaches to detect different types of RNA variation and significantly expands understanding of the transcriptome's diversity.

NOMAD is a highly efficient algorithm that operates directly on raw sequencing data to identify differentially diversified sequences, characteristic of some of the most important transcript regulation such as splicing, RNA editing, V(D)J recombination. NOMAD achieves this by parsing reads into k-mer sequences, called anchors, that are followed by diverse sequences, called targets, a fixed distance downstream of them. NOMAD calls anchors that have sample-specific target expression, reflecting inter-cell variation in expression that can be signature of alternative RNA splicing, RNA editing, and among many other examples. Anchors are too short (selected as 27-mers by default) for a biological interpretation of underlying mechanism generating sequence diversity. To fill this gap, NOMAD was expanded to include a new algorithm that functions directly on raw sequences to provide sensitive, seed-based branched local de novo assembly (FIG. 7A), referred to as NOMAD+.

Each significant anchor sequence is used as a seed in the assembly step. First, fastq files are parsed for each called anchor and the reads containing each anchor are collected. For each position downstream of the anchor with multiple observed nucleotides, the local assembly potentially branches into multiple sequences using a simple statistical criterion, depending on the number of nucleotides with frequencies exceeding a fixed threshold. This rule is applied recursively, resulting in extended anchors called “compactors” whose length is at most the input read length and required to have raw read support (FIG. 7A). After this step, each called anchor is associated with a set of compactor sequences along with the set of reads assigned to each compactor during its branching process. Compactors can be thought of as a simple seed based micro assembly.

Compactors denoise input reads and enable discrimination of splice isoforms, editing events, V(D)J recombinants and sequences outside of the human reference. Unlike any other de novo transcript assembly in use for scRNA-seq, compactors can be statistically analyzed to quantify the probability that reads supporting an artifactual compactor will be observed. Compactors also enable huge reductions in computational burden for the number of sequences analyzed in any downstream analysis such as alignment to the genome. In this study, compactors reduced the number 120-fold: from 183,471,175 raw reads to 1,515,555 compactors.

NOMAD+ also includes a classification procedure operating on compactors to assign NOMAD's calls to biologically-meaningful categories such as splicing, among many other categories (FIG. 7B), improving interpretability of the NOMAD+ calls and facilitating either targeted or integrative downstream analysis on the anchors within and across multiple categories. Each anchor is classified based on its two most abundant compactors into 6 different categories: splicing, internal splicing, base pair change, 3′UTR, centromere, and repeat. This classification is based on both computing edit distance between the two compactors for each anchor, and also mapping these compactors to the T2T genome using a spliced aligner (e.g., STAR (Dobin et al. 2013, cited supra)). An anchor is classified as splicing if STAR provides a spliced mapping for at least one of the compactors. When both compactors lack splice junctions, the anchor is classified as internal splicing or base pair change according to the string edit distance between the compactors. The mapping positions for the remaining unclassified anchors are further intersected with annotation databases for 3′ UTRs, centromeric repeats, and repeats to classify to one of these categories accordingly. Additionally, soft-clipped portions of the compactor are realigned to the reference genome to allow for putative transposable elements, circular RNA, and other intra-, inter- or extra-genic transcription. Through this classification, each compactor becomes part of a family of compactors defined by their shared anchor. Therefore, for each anchor, compactors that fail to map can still be annotated by the annotation of the most abundant compactor for that anchor, which was referred to as annotation by association described below. Together, compactors enable interpretation of transcript diversity that can bypass the biases introduced by reference-first approaches, increase the interpretability of calls, and allow a direct comparison of NOMAD+ to existing algorithms: by using references only for interpretability rather than for statistical inference, NOMAD+ obtains statistically valid and unbiased inference, as well as interpretable results.

### NOMAD Overview

NOMAD is a reference-free, annotation-free method that can be directly applied to raw sequencing reads and provide a unified statistical approach for the (co)detection of various transcript diversification mechanisms including (nut not limited to): alternative splicing, RNA editing, V(D)J recombination, and chimeric RNAs such as gene fusions, inversions, and circular RNAs. Not requiring computational alignment of the reads to a reference genome, a feature commonplace in conventional methods for RNA expression analysis, NOMAD can bypass inherent biases and blindspots in aligners leading to discoveries not possible by other methods. NOMAD searches for sequences of certain length (anchors) which are followed by diverse sequences (targets). Then for each extracted anchor, a contingency table containing the read counts for each target and each sample is generated. NOMAD then performs a statistical test with closed-form solution for valid p-value to find anchors with significant sample-dependent target count distribution. The test statistic is constructed through random partitioning of the samples, and using random hash functions to map each target to a random value in {0,1}. P-values for each anchors are corrected for multiple testing across generated random partitions (L_num_random_Cj) and number of generated random hashes for each partition (K_num_hashes). P-value for each anchor is corrected for multiple testing across the number of partitions and hashes.

### NOMAD Runs

NOMAD was run in unsupervised mode on the fastq files from each donor and tissue in Tabula Sapiens data set. 19 tissues and 12 donors from the Tabula Sapiens dataset (Tabula Sapiens Consortium, et al. 2022, cited supra) that have been profiled by SmartSeq2 were used for the analysis. 400 annotated cells with cell type information were randomly selected from a tissue and donor. Eight donor-tissues had fewer than 400 cells: Trachea TSP2 (119 cells), Eye TSP5 (134 cells), Blood TSP1 (138 cells), Tongue TSP4 (209 cells), Heart TSP12 (277 cells), Eye TSP3 (291 cells), Trachea TSP6 (358 cells), Kidney TSP2 (370 cells). Because 400 cells were sampled for each donor and tissue (except 8 tissues with between 119 and 370 cells), cell number normalization is implicit, and read depth is approximately so. In total, NOMAD was ran on 13,500 SmartSeq2 cells from 136 cell types. NOMAD was run with default parameters except for the following parameters for number of random partitions for input cells and number of random hashes for each partition: L_num_random_Cj=300 and K_num_hashes=10.

Anchors with |NOMAD effect_size|>0.2, target Levenshtein distance>1, number of reads assigned to the anchor>50, and observed in >10 samples (cells) selected for compactor generation and downstream analysis.

### Compactor Generation

Reads containing significant anchors are aggregated across FASTQs, and read segments upstream of the anchor sequence are discarded. For each anchor, reads are traversed left-to-right and nucleotides at each position are tested for support in the reads. If at least 20 reads present a particular nucleotide and the read number exceeds 10% of reads on the current branch (or 5 reads and 80%), then reads containing this nucleotide are branched to be traversed and tested independently, and this nucleotide is appended to their representative compactor sequence. This rule is applied recursively, resulting in subsets of all anchor-reads each represented by a distinct compactor sequence.

All STAR-unaligned compactor sequences were submitted to hmmsearch for alignment to the Pfam and to cmscan search of the Rfam database. For BLAST, the following two compactor subsets were produced for each anchor:

First collect the set of sequences which have been unaligned by STAR.

If the anchor has 100 or more compactors, take the 10 most abundant compactors to be BLAST-aligned. If the anchor has fewer than 100 compactors, take the 2 most abundant compactors to be BLAST-aligned.

Then take the union of these subsets and submit to BLAST with the following parameters:

-evalue 0.1 -dust no -word_size 24 -reward 1 -penalty -3 -max_target_seqs 4
Reference-Free Classification of Compactors into Biologically-Relevant Categories

To increase interpretability of the inferred compactors and to be able to perform targeted downstream analysis for a specific RNA diversity event, an RNA event was assigned to each anchor based on its compactors and the features directly derived from the compactors. Six categories were considered for anchors: splicing, internal splicing, base pair change, 3′UTR, centromere, and repeat. If an anchor is not assigned to any of these categories, it will be categorized as unclassified. A hybrid approach was utilized for assigning classes to the anchors, where some classes are assigned independently from the alignment to a reference genome (e.g., internal splicing and base pair change) and some classes are assigned based on the reference genome (e.g., splicing, 3′UTR, centromere, and repeat). As each anchor might be qualified for more than one class, classes were prioritize in the following order: splicing, internal splicing, base pair change, 3′UTR, centromere, repeat.

To classify anchors, only the top two most abundant compactors were considered (i.e., those with the highest fraction of anchor reads) for each anchor. If one of the compactors is longer than the other one, then it is examined to consider if its substring is of the same length as the other compactor. Two different distance metrics were computed: hamming distance and Levenstein distance. Both strings should be of the same length to be able to compute Levenstein distance. Both Levenstein and hamming distance are computed when the anchor sequence is removed from the beginning of each compactor sequence. Anchors with the same hamming and Levenstein distances are classified as mutations as this criterion indicates that only substitutions (i.e., nucleotide changes) can explain the difference between the compactors.

If the Levenstein operations sequence for an anchor includes a number of consecutive insertions and deletions, the anchor is classified as “internal splicing” if: Levenstein_distance<(run_length_D+run_length_I+1), where run_length_D and run_length_I are the longest stretch of deletions and insertions in the Levenstein operations sequence, respectively.

### Reference-Based Classification

To find anchors that can be explained by an alternative splicing, the two compactors for each anchor were mapped to the reference genome using a spliced aligner (e.g., STAR, but any other splice aligner could be used). Compactors were aligned to the reference human genome T2T using STAR in two-pass mode with the following parameters:

--twopassMode Basic --alignlntronMax 1000000 --chimJunctionOverhangMin 10 --chimSegmentReadGapMax 0 --chimOutJunctionFormat 1 --chimSegmentMin 12 --chimScoreJunctionNonGTAG -4 --chimNonchimScoreDropMin 10 --outSAMtype SAM
--chimOutType SeparateSAMold --outSAMunmapped None --clip3pAdapterSeq AAAAAAAAA
--outSAMattributes NH HI AS nM NM

Information about the mapping flag, mapping chromosome, mapping coordinate, CIGAR string, and number of mismatches from STAR BAM file (1st, 2nd, 3rd, 4th, 6th, and 16th columns) was extracted. If at least one of the compactors for an anchor involves a split mapping and the hamming distance and Levenstein distance are not equal, the anchor was classified as “splicing”, as at least one of the compactors involves a splice junction and the difference between the compactor sequences cannot be explained by simple substitutions. Note that both compactors should overlap to the same gene.

For human genome, for anchors that have not been classified as splicing, base pair change, and internal splicing, compactor mapping positions was further intersected with the 3′UTR coordinates, centromere satellite element coordinates in T2T CenSat database and repetitive element coordinates in RepeatMasker database and classify anchors to one of these categories accordingly.

### Soft-Clipping and Realignment

Compactors that have been aligned by STAR through softclipping can provide evidence for RNAs not part of the reference transcriptome such as circular RNAs, gene fusions, and strandcrosses. As a systematic approach for utilizing compactors with soft-clipping to infer such RNAs, those compactors with >20 soft-clipped bases in which the longest stretch of any nucleotide A, G, C, T is shorter than 6 and realign the softclipped part to the genome using STAR were selected. The information from the original alignment of the compactor with the realignment of its softclipped part was used to infer if the compactor can provide evidence for circular RNA, gene fusion, and strandcross.

For a softclipped compactor to be classified as circular RNA, the mapping positions and the strand orientation of the original compactor and softclipped part were examined. Let m_C and m_S be the mapping positions for the compactor and its softclipped part, respectively, and also s_c and s_s be the strand orientations for the compactor and its softclipped part, respectively. Assuming that the softclipped part is at the start of the compactor, a compactor was classified as circular RNA, if it satisfies one of the following conditions and both alignments map to the same chromosome:

$$( s\_c == + ) \& ( s\_s == + ) \& m\_s > m\_c$$

$$( s\_c == - ) \& ( s\_s == - ) \& m\_s < m\_c$$

Similarly, if the softclipped part is at the end of the compactor, one of the following conditions should be met by a compactor to be classify as a circular RNA:

$$( s\_c == + ) \& ( s\_s == + ) \& m\_s < m\_c$$

$$( s\_c == - ) \& ( s\_s == - ) \& m\_s > m\_c$$

To assign strandcross to a compactor, both alignments should have the same chromosomes but different strand orientations. Finally, for gene fusion compactors, either the mapping chromosomes for the compactor and its softclipped part are different or they map to the same chromosome and strands but with a distance of at least 10\hat{ }6 bases. Note that for a compactor to be assigned to one of these classes, both the compactor and its softclipped part should be uniquely mapped by STAR.

### Supervised Compactor Generation

NOMAD-called anchors from a single donor-tissue can be missing from the NOMAD calls in other donor-tissues. This can be caused by NOMAD's downsampling of input FASTQs and by inconsistencies in an anchor's signal between donor-tissues. To investigate anchor sequences called in one donor-tissue but not another, NOMAD-called anchors were used as seeds for compactor generation in donor-tissues where NOMAD did not call the anchor.

### Classification of Anchors by Alignment to Reference Databases

Anchors were assigned to categories by their alignment to a set of reference databases. Bowtie2 was used to align anchors to references; default parameters were used. Anchors are categorized by their top annotation, reported according to the following priority: false positive sequences (such as Univec, Illumina adaptors), Rfam, transposable elements (Dfam), spacers, and the human genome (hg38).

### Aggregation of Anchors Abundance Over Cell Types

To quantify anchor abundance on the cell type level, a compactor output file, which contains per-sample counts for each anchor-compactor, was used. The sample identities are converted to cell type values, with the use of cell-level metadata. Counts are then summed, to provide aggregated counts per anchor-cell type. Anchors are converted to their compactor classification values and further aggregated via summation, to provide counts per classification-cell type.

Computation of Compactor p-Values

The same contingency table test was additionally utilized on the compactor x sample contingency table, to generate a statistically valid p-value bound testing whether the distribution of compactors is the same across samples or not. A similar procedure to that was utilized for generating the initial p-values on the target x sample count matrices, selecting 50 pairs of random c and f, and taking the minimum p-value across these random c and f after applying Bonferroni correction. The analysis techniques are identical to those used in the original NOMAD. Alternating maximization-based c and f was additionally utilized, where p-values are derived using a sample splitting approach, recently derived in.

Statistically valid p-value bounds were provided for observing large counts of the third most abundant target under a 2-target null hypothesis. For M reads, with sequencing error rate epsilon (each basepair independently undergoes a substitution error with probability epsilon, uniformly erroring to one of the other 3 basepairs). DKL(p,q) denotes the Kullback-Liebler divergence between two independent Bernoulli random variables with heads probabilities p and q. Here, the probability of observing more than T counts of the third most abundant length k target was bounded.

$$2 {\sum }_{ℓ = 1}^{k} ( \begin{matrix}k \\ ℓ\end{matrix} ) {3}^{ℓ} exp ( - M {D}_{KL} ( \frac{T}{M} \vert \vert {( 1 - ϵ )}^{k - ℓ} {( ϵ / 3 )}^{ℓ} ) )$$

This bound decays very quickly as a function of T. For example, for M=50 and epsilon=0.01, T=4 yields a p-value of 0.01, T=5 yields a p-value of 1 E-5, and T=7 yields a p-value of 1 E-10. This can naturally be extended to bounding the probability of observing many counts for the j+1 st target, given that only j targets truly exist without errors.

### Robustness to Biochemical Sampling Error Models

After biochemical sampling, counts in single cell RNA-seq are often over-dispersed. Thus, counts are often modeled as poisson with a random (gamma distributed) mean, which is equivalent to the widely used negative binomial distribution. This has been studied in DESeq2 (M. I. Love, et al., Genome Biology 15 (12): 550, the disclosure of which is incorporated herein by reference), where the authors show that the overdispersion can be well modeled as a function of the sequencing depth, where low sequencing depth leads to higher dispersion than would be expected under the poisson null. NOMAD's robustness was simulated to overdispersion, showing that under the overdispersion modeled by DESeq2, NOMAD provides much better control of the false discovery rate against this biological null (as opposed to the statistical null), whereas a classical chi-squared test immediately begins to aggressively reject once the null is no longer satisfied (dispersion>0). For example, for 20 observations per sample, with 10 equally likely targets and 20 samples, NOMAD still controls the FDR below 5% while the chi-squared test has an FDR of approximately 87%.

### Example 6: NOMAD+ Improves Precision of Spliced Calls and Identifies Extensive Splicing in CD47 Including Novel Isoforms

SPLICING More than 95% of human genes are known to be alternatively spliced, but the number of dominant expressed isoforms is mainly based on bulk tissue-level analyses and continues to be debated. This debate is based on alignment-first and reference-based approaches, many focused on specific tasks such as detecting linear alternative splicing, performing metadata (e.g., cell type) guided testing, and approximate statistical inference due to problems associated with mapping to multiple isoforms.

NOMAD+ was tested to determine whether a statistics-first approach could lend clarity to this debate. NOMAD+ reported 20,385 anchor calls classified as splicing across all donors and tissues, including 11,995 and 3,700 unique anchor sequences and genes, respectively. First, robustness to biochemical sampling obstacles was evaluated, including dropouts and PCR bias present in SS2 libraries that can be approximated with the negative binomial probability distribution. Under sampling counts from negative binomial distribution identical for each sample which formally violates the null hypothesis of targets are drawn from a distribution independent of cell identity but does not represent real biological signal, Pearson's chi-squared, a widely-used classic statistical test of target-sample independence, calls a high fraction of false positives (FDR>80% at a nominal FDR of 5%), while NOMAD+ retains a maximum FDR of 5%. NOMAD's robustness was also evaluated by recovery of annotated splicing events and reproducibility of calls in the same tissue but between different donors.

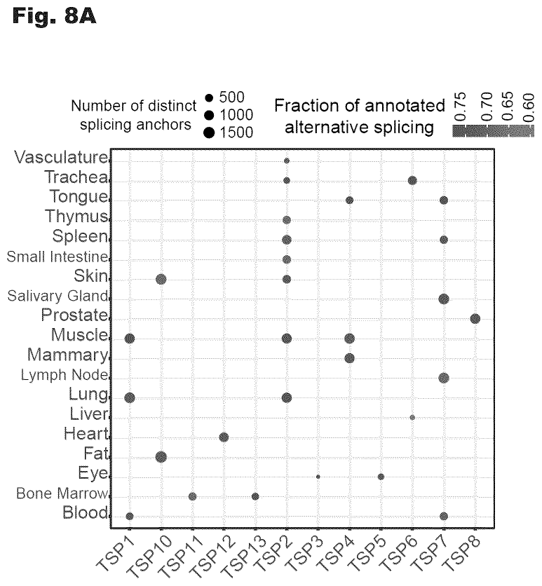

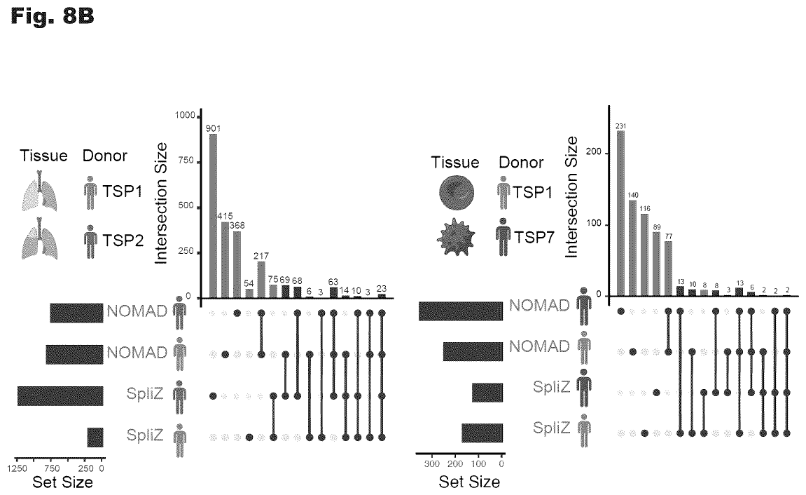

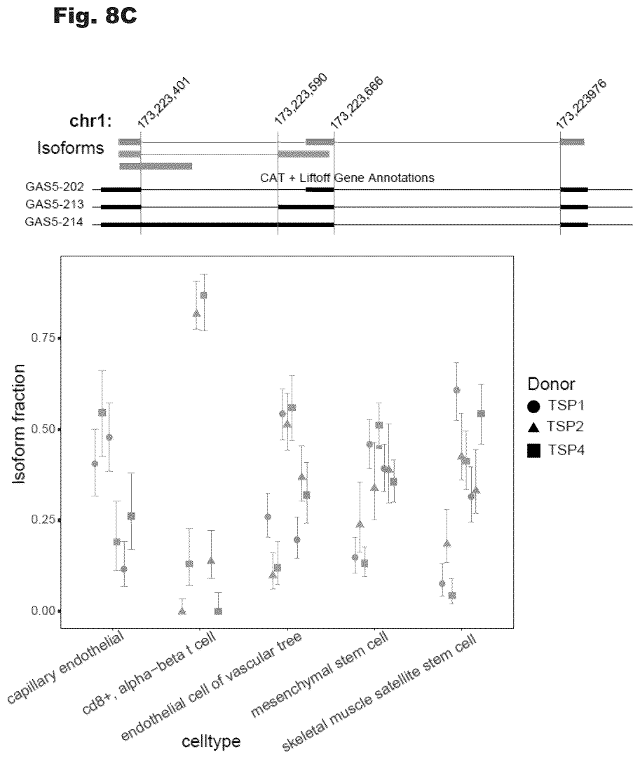

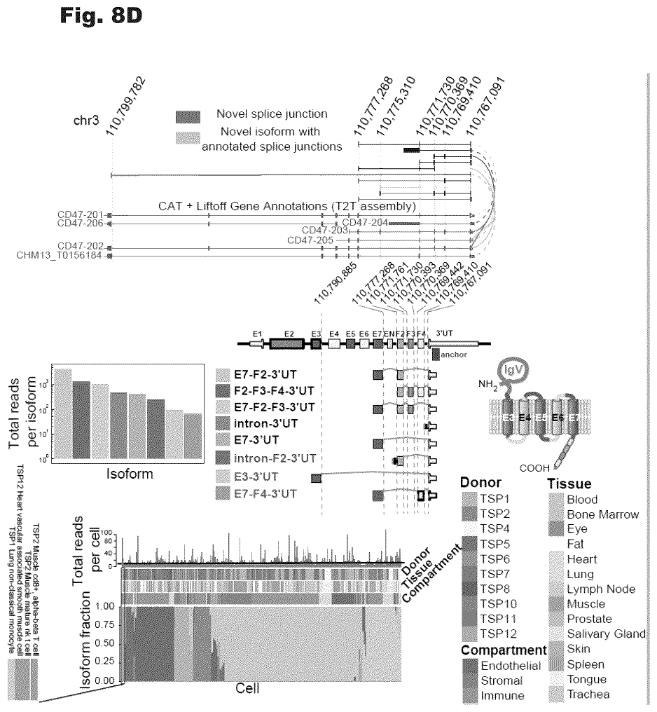

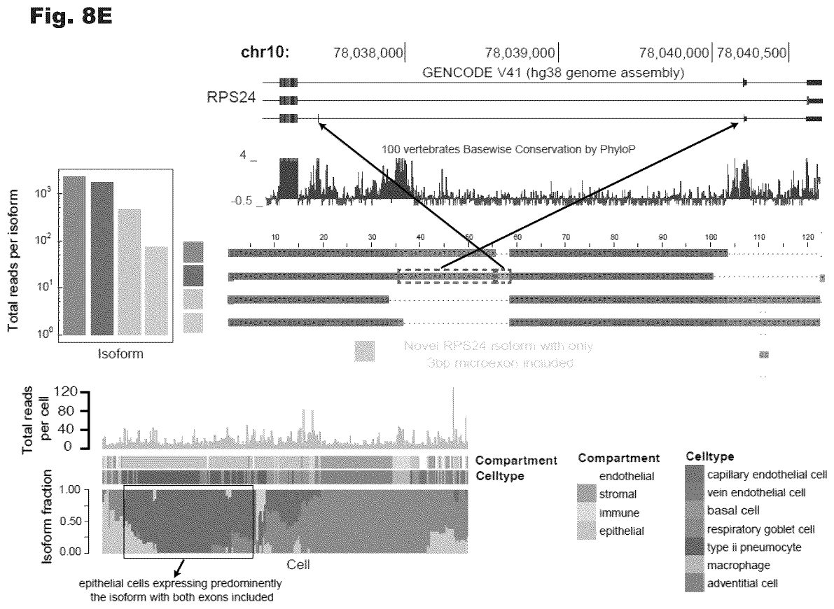

73.2% of anchors classified as splicing had compactors mapping to annotated alternative splicing junctions (FIG. 8A). A minority (7.5% or 1,537 anchors) had compactor sequences mapping to an unannotated junction, including 1,387 anchor calls with >10% reads mapping to the unannotated junction. 706 of these anchors found in more than one donor-tissue pair. NOMAD+ improved the reproducibility and precision of the current best performing algorithm for detection of single cell regulated alternative splicing, SpliZ, and predicted complex splicing patterns missed by this method. This improvement by NOMAD+ is noteworthy: it did not use any cell type metadata and was run on subsamples of these tissues, not matched for cell type composition, and thus tissue-donor samples are not formally biological replicates. Focusing on the lung, blood, and muscle—where >1 donor was available, NOMAD+ achieved higher concordance for the same tissue between different donors (FIG. 8B, Supplement). In blood and lung, NOMAD+ showed significantly higher reproducibility between donors compared to SpliZ: 77/501 (15.4%) compared to 9/272 (3.3%) for blood; and 202/1250 (16.2%) compared to 75/1289 (5.8%) for lung (FIG. 8B). For muscle, both NOMAD+ and SpliZ called almost the same number of unique genes across 3 donors; however, 111 NOMAD+ calls were shared across all three donors, versus only 17 were shared by SpliZ. The most highly expressed anchor found in all three muscle replicates and missed by the SpliZ was GAS5, a noncoding RNA regulating apoptosis and growth. GAS5 shows reproducible cell type- and compartment-specific alternative splicing (FIG. 8C).

Recent reference-based metadata-guided studies on human cells and experimental validations have found that MYL6 and genes in the TPM family undergo highly cell type-specific alternative splicing (J. E. Olivieri, et al., bioRxiv. doi.org/10.1101/2021.05.01.442281 (2021), the disclosure of which is incorporated herein by reference. All these genes were also found by NOMAD+ highlighting its power for detecting cell type-specific patterns even without cell metadata. NOMAD+ re-identified regulated splicing patterns and extended findings to reveal combinatorial expression of isoforms. In muscle, true positives MYL6 and TPM1 were significant in all three donors; in contrast, SpliZ only called TPM1 and MYL6 in donors 1 and 2. Both NOMAD+ and SpliZ called TPM2 and TPM3 in only donor 4.

CD47, a clinical target for both cardiovascular events and cancer immunotherapy, was also investigated as previous work showed CD47 isoform expression was compartment-specific (see J. E. Olivieri (2021), cited supra). Among all CD47 anchors classified as splicing, NOMAD+ detected 10 distinct spliced isoforms, including 5 novel isoforms (FIG. 8D). One anchor reveals expression of 8 distinct isoforms including 2 novel isoforms (FIG. 8D), all impacting either the cytoplasmic or transmembrane domains. Compartments prefer different isoforms: endothelial and stromal compartments predominantly express E7-F2-3′UT isoform, while immune and epithelial cells in addition to this isoform also express F2-F3-F4-3′UT and E7-F2-F3-3′UT isoforms, respectively. One of the novel isoforms detected is denoted intron-F2-3′UT (FIG. 8D); if this isoform indeed represents full intron retention, it would also result in a stop codon after the first transmembrane domain, similar to E7-3′UT isoform, a second novel prediction.

NOMAD+ revealed new insights into splicing of RPS24, a highly conserved essential component of the ribosome; annotations show >5 annotated isoforms that include ultraconserved intronic sequence and microexons (see J. E. Olivieri (2021), cited supra). NOMAD+ detected 4 isoforms in lung cells from donor 2, respecting the previous findings of compartment specificity for this gene. However, they are also extended, with NOMAD+ identifying a novel isoform containing only a microexon which both STAR and current annotation miss (FIG. 8E). Together, this analysis shows that without using any cell metadata or reference before significance calls are made, NOMAD+ has greater sensitivity and reproducibility than SpliZ

### SpliZ Runs

SpliZ is a statistical method for detecting genes with cell type-specific alternative splicing in scRNA-Seq (J. E. Olivieri, et al., bioRxiv. doi.org/10.1101/2021.05.01.442281 (2022), the disclosure of which is incorporated herein by reference). It assigns a single score to each pair of cell and gene and is reference-dependent in the sense that it needs the split reads mapping to the splice junctions of the gene to compute. To compare NOMAD with SpliZ (as a state-of-the-art reference-dependent method), SpliZ was run on the same Tabula Sapiens dataset. Reads were aligned to human hg38 reference genome using STAR and then ran the STAR BAM files through SICILIAN, which is a statistical wrapper for detecting high-confidence splice junctions from spliced aligners (R. Dehghannasiri, et al., bioRxiv. doi.org/10.1101/2020.04.14.041905 (2020). SpliZ was then applied to the detected splice junctions. SpliZ was ran on data from each donor separately and to avoid calling genes that have tissue-specific splicing rather than cell type-specific splicing, its statistical test was performed separately across cell types within each tissue from that donor.

### Comparison to STAR and SpliZ

After running SpliZ and obtaining the list of genes with cell type-specific splicing for each donor and tissue called by SpliZ, the list of significant genes found by NOMAD and SpliZ were compared for each donor and tissue separately. Since NOMAD calls are the anchor-level and not gene-level to have consistent comparison with SpliZ whose calls are at the gene-level, it was assumed that a gene is found to be significant by NOMAD if it calls at least one “splicing” anchor for that gene.

### Example 7: NOMAD+ Rediscovers and Expands the Scope of V(D)J Transcript Diversity

VDJSingle cells can somatically acquire copy number variation, SNPs, or repeat expansions. Detection of genetic diversity in single cells has required custom experimental and computational workflows. NOMAD+ unifies this discovery by a hypothesis testing framework: under the null, all cells in any donor have only two alleles of any fixed splice variant. Under this null, at most two compactors sharing a splice junction should be observed. This framework was used to validate NOMAD+ calls and prioritize its predictions of transcriptional novelty. Positive controls expected to violate the null include mitochondria where genomes are polyploid, as well as the rearrangement of immunoglobulin loci in B cells and T cell receptor loci which undergo V(D)J recombination, among other examples. Other events also expected to violate the null include post-transcriptionally generated variation, such as RNA editing or repeat expansions.

To investigate global trends, for each anchor, distinct compactors were found across all donor-tissues. Genes annotated as immunoglobulin had the highest number of compactors. An anchor mapping to immunoglobulin kappa chain (IGKC) has the greatest number of compactors—140—across all donors and tissues of any gene. Interestingly, these anchors were observed in all immune tissues. Other immunoglobulin genes were also enriched for high numbers of compactors and differentiated from other genes on these purely numerical criteria. Centromeres are thought to have high sequence diversity within and across individuals, thus are expected to have high compactor number across tissues: centromeric anchors defined as containing CCATTCCATT (SEQ ID NO: 6) or their reverse complements, have highest numbers of compactors across donors and tissues. Mitochondrial genes also had high diversity, highest in an anchor with compactors mapping to MT-ND5, with 24 compactors in donor 1 lung, the greatest number of compactors generated in a single donor-tissue by any anchor with compactors mapping to an annotated gene. MT-ND5 is a component of the transmembrane electron transport chain in mitochondria with previously reported recurrent mutations with clinical significance.

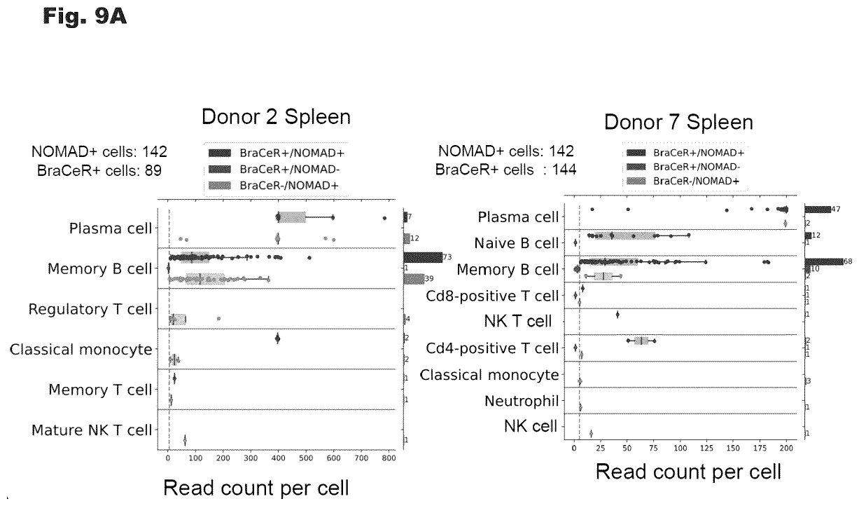

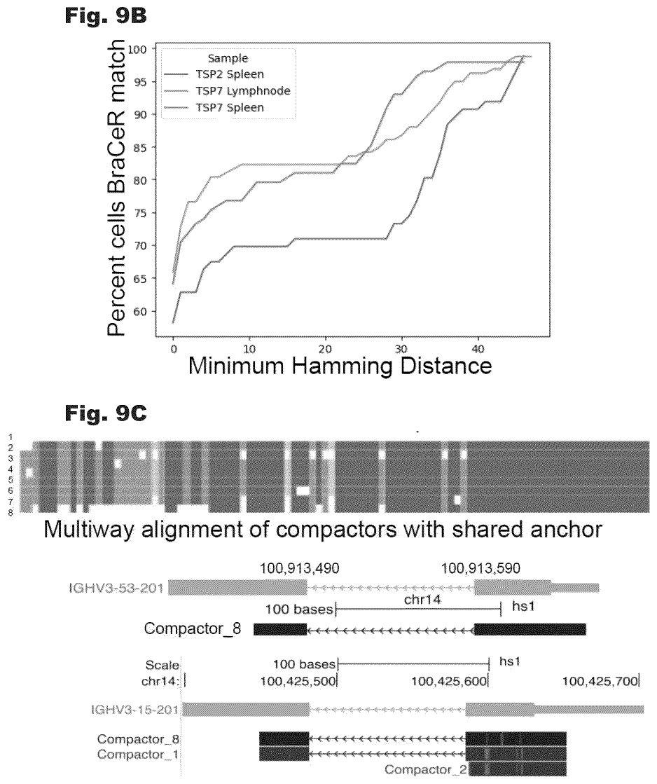

While current approaches require mapping to the genome on a read-by-read basis, NOMAD+ enables a statistics-first micro-assembly to detect variants in the B cell receptor (BCR) locus avoiding genome alignment. First, it was evaluated whether NOMAD+ discovered BCR rearrangements, which was referred to as “IG-compactor” and defined as a compactor mapping to gene with an immunoglobulin annotation and matched those found by the state-of-the-art custom pipeline BraCeR (I. Lindeman, et al., Nature Methods 15 (8): 563-65 (2018), the disclosure of which is incorporated herein by reference). In donor 2 and donor 7 spleen, NOMAD+ detected an IG-compactor in every cell which BraCeR reported a BCR contig, as well as additional cells which BraCeR missed. For example, in spleen plasma B cells from donor 2, not only did NOMAD+ detect IG-compactors in all the 7 cells with BraCeR calls, but it also found IG-compactors in 12 additional cells. Similarly, in donor 7 spleen BraCeR and NOMAD+ found evidence of BCR rearrangement in the same 47 plasma B cells, but NOMAD+ found IG-compactors in 2 additional plasma B cells which BraCeR did not (FIG. 9A). There are instances where NOMAD+ is less sensitive than BraCeR, such as in spleen memory B cells from donor 7, where BraCeR and NOMAD+ both found BCR evidence in the same 68 cells, but NOMAD+ misses 10 cells which BraCeR calls due to NOMAD+'s requirement that IG-compactors be supported by at least 5 reads.

Out of the cells analyzed by both algorithms, NOMAD+ called IG-compactors in 142 cells from donor 2 spleen and 142 and 123 in donor 7 spleen and lymph node. It was tested to determine if NOMAD+'s IG-compactors were concordant with BraCeR's calls in these cells by computing the minimum Hamming distance between the two sets of BraceR contigs and NOMAD+IG-compactors for each cell. A high fraction of cells have perfect matches to BraCeR's calls in the same cell: 58.1%, 65.8%, and 64.1% for donor 2 spleen, donor 7 spleen, and donor 7 lymph node respectively, with increasing concordance as for more relaxed minimum Hamming distance criterion for calling a match between IG-compactors with BracerR calls (FIG. 9B).

NOMAD+ calls missed by BraCeR were investigated, further restricting such candidate compactors to have a minimum Hamming distance of greater than 30 bps to all BraCeR contigs, as well as requiring 3 or more compactors per anchor, with either 20 soft-clipped bases, split-mapping, or >4 mismatches to the genome to support their being called due to rearrangement and or hypermutation. NOMAD+ detected 416 anchors with IG-compactors with the above stringent qualities. Another anchor contained 8 compactors, which each aligned perfectly to different IGHV loci, likely representing distinct V segment inclusion. The alignment of one of these compactors is shown as well as the sequence similarity between all eight compactors of the anchor (FIG. 9C). In summary, NOMAD+ automatically detects V(D)J rearrangement agreeing with, but extending that detected by BraCeR in expected B cell subtypes, with implications for downstream biological inference and opportunities to explore other sequences nominated by NOMAD+ that do not meet the stringent criteria used here.

### NOMAD+IG-Compactor Comparison to BraCeR

To test whether NOMAD+ was detecting IG-compactors with perfect sequence similarity to BraCeR contigs from the same cell, the minimum Hamming distance between all IG-compactors and all BraCeR contigs was calculated. IG-compactors are defined as NOMAD+ compactors which map to an immunoglobulin heavy, light, or kappa chain gene by STAR, allowing for mismatches and soft-clipping. IG-anchors were further defined as anchors which have a plurality of compactors (at least 20%) mapping to immunoglobulin genes. IG-anchors allow for annotation-by-association compactors which are unmapped by STAR, but have the same anchor as predominantly IG-mapping compactors. The minimum Hamming distances of all IG-anchor compactors were calculated against all BraCeR contigs downloaded for the same donor from the Tabula Sapiens AWS bucket, where the * is 2 for donor 2 etc.

Setting BraCeR contigs as ground truth, the true-positive and false-positive rates (TPR and FPR) of NOMAD+ were estimated by constructing a square binary matrix M, identically indexed on rows and columns by the cell-ids that were run through both NOMAD+ and BraCeR. The i,j entry in the M matrix is 1 if there is a perfect alignment, Hamming distance 0, of at least one compactor from cell-i to any of the BraCeR contigs from cell-j, otherwise M[i,j]=0. From this table the TPR was defined as the sum of the diagonal entries of M, M[i,i], divided by the number of diagonal entries, which is again the number of cells run through both BraCeR and NOMAD+. Similarly, the FPR is the sum of off-diagonal entries divided by the number of off-diagonal entries. Note that if a single compactor has perfect alignment to multiple contigs, or vice-versa. each of those pairs will contribute to either the TPR or FPR.

Another category of IG-compactors was defined as “interesting” if the IG-compactor was a more stringently filtered subset of IG-compactors. It was required that the compactor is (i) IG-compactor (ii) IG-anchor (iii) has 10 or more compactors unique per anchor (iv) has a total anchor abundance greater than 100 (v) must be STAR aligned (vi) has a minimum Hamming distance larger than 30 to BraCeR contigs, and finally (vii) has either more than 20 soft-clipped bases, split-mapping, or has more than 5 mutations with respect to the reference. These interesting IG-compactors were used to test if NOMAD+ was extending past BraCeR by discovering BCR recombination events in cells which did not have BCR contigs. When calculating the TPR and FPR above it was ensured that the IG-compactors and BraCeR contigs were only counted if they had perfect sequence similarity, but for this analysis to test NOMAD+ sensitivity beyond the BraCeR calls, sequence comparisons were not made. Instead a cell was called BraCeR+/NOMAD+ if it contained at least one BraCeR contig and at least one interesting IG-compactor with 5 or more reads in that cell.

### Example 8: NOMAD2

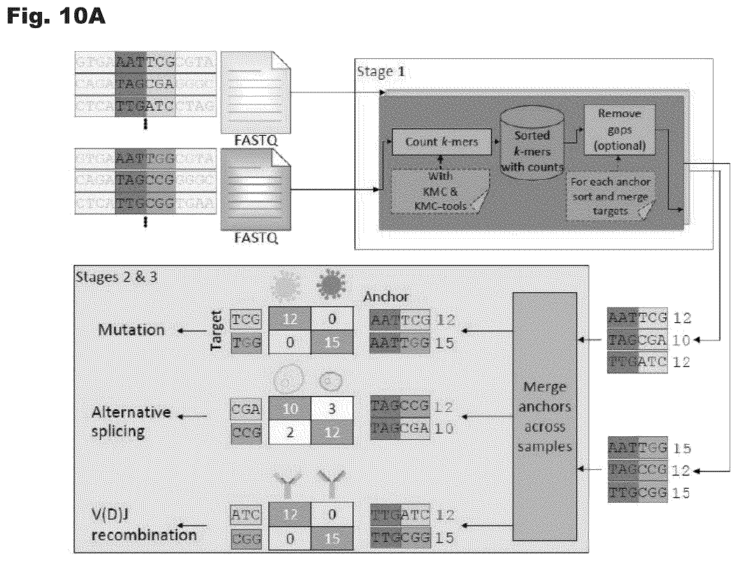

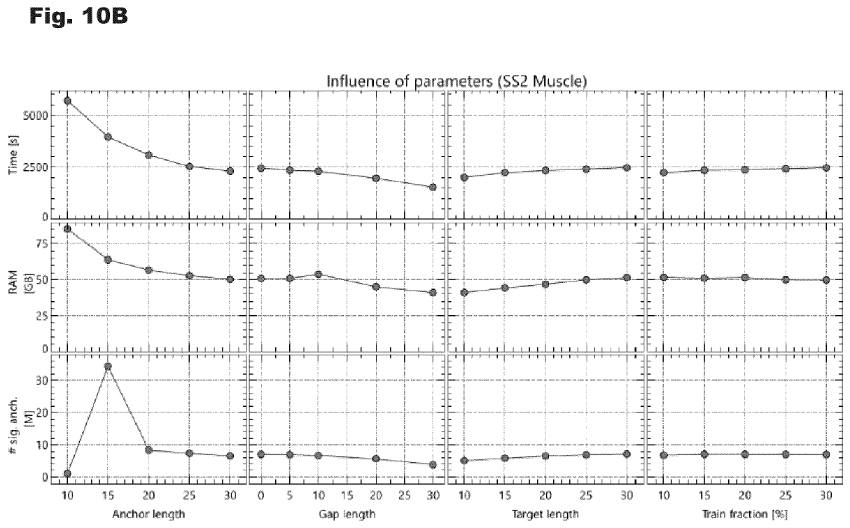

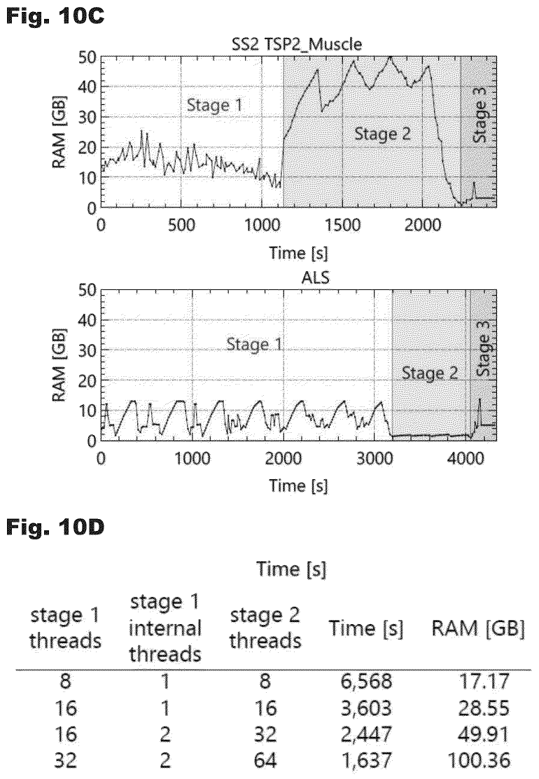

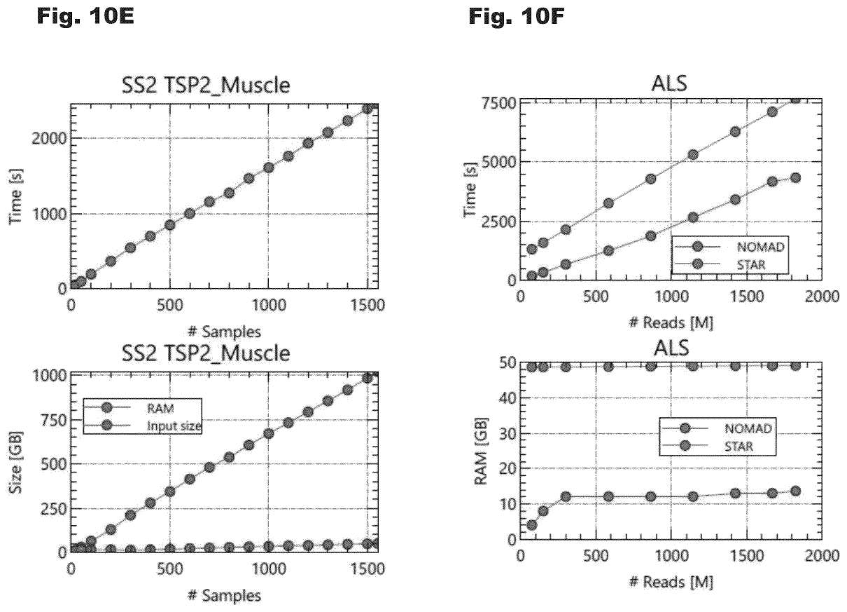

This example provides another statistical methodology: NOMAD2 and illustrate a snapshot of its novel biological discovery in three massive datasets: 1,553 single cell RNA-seq samples (Tabula Sapiens Consortium, et al., (2022), cited supra), 671 cancer cell lines (M. Ghandi, et al., Nature 569 (7757): 503-8, the disclosure of which is incorporated herein by reference), and deeply sequenced patient samples from ALS (X. Ma, et al., Nature. 603:124-130 (2022), the disclosure of which is incorporated herein by reference). NOMAD2 is implemented in 3 stages (FIG. 10A). In stage 1, NOMAD2 records all k-mers (substrings of length k) containing anchor, target, and counts in each given sample separately with KMC (see, S. Deorowicz, et al., BMC Bioinformatics. 4:160 (2013); and M. Kokot, et al., Bioinformatics 33 (17): 2759-61 (2017); the disclosures of which are incorporated herein by reference). KMC and its alternatives, e.g. Jellyfish (G. Marcais and C. Kingsford, Bioinformatics 27 (6): 764-70 (2011), the disclosure of which is incorporated herein by reference), DSK (G. Rizk, et al., Bioinformatics 29 (5): 652-53 (2013), the disclosure of which is incorporated herein by reference) do not allow any gaps in the counted substrings, and so do not directly enable the generality of NOMAD's analytic framework. First, NOMAD2 enumerates all (a+g+t)-mers (a, g, t being anchor, gap, and target lengths, respectively). In the second step, these sequences are sorted lexicographically with KMC-tools. As a result, all occurrences of unique anchors are adjacent, which enables efficient gap removal and unique targets collapsing in the third step via a linear traversal over the (a+g+t)-mers which collects all targets for the current anchor (gaps are skipped). Then, targets are sorted and the unique targets merged (counts are updated appropriately) producing the records requested at this stage. The memory footprint of this stage is low, mainly thanks to the memory-frugal KMC and KMC-tools employed here.

In the second stage, NOMAD2 reads all the lists of records linearly to merge the anchors across samples and outputs a list of all anchors with associated summary statistics such as effect size. For each anchor the results are merged in a list of tuples (containing target, sample id, and count). Each list is used to construct a contingency table, a base data structure used to compute a statistically valid p-value, along with several measures (e.g. effect size) for downstream interpretability. This p-value is constructed using unsupervised optimization (see T. Baharav, et al., (2023), cited supra). This step is also memory-frugal (FIG. 10C), because only data of a single anchor needs to reside in main memory, and is represented as a sparse matrix.

The third stage loads all anchors with their computed p-values into main memory to perform multiple testing correction, to yield an FDR controlled list of significant anchors (see Y. Benjamini and D. Yekutieli, The Annals of Statistics 29 (4): 1165-88 (2001), the disclosure of which is incorporated by reference. Memory consumption is low as there is now a single record for each anchor (its p-value) (FIG. 10C). The majority of NOMAD2's code is parallelized.

Performance of NOMAD2 was evaluated on a machine equipped with AMD 3995WX 64-Cores CPU, 512 GB RAM, and 4 HDDs configured in RAID5 using two datasets ALS (15 samples, 1.82 G reads, 550 GB FASTQ), and SS2 Muscle (1,553 cells, 3.69 G reads, 1.02 TB FASTQ). Experiments revealed that most of the crucial parameters have little impact on the running time/RAM usage/number of significant anchors detected, with the exception of moderate values of anchor length (those between 10 and 20, FIG. 10B). Depending on the dataset, stage 1 or stage 2 is the most resource-demanding, where stage 3 requires <10% of the total running time (FIG. 10C). Increasing the number of threads decreases running time at the cost of increased memory consumption (FIG. 10D). Memory usage and runtime grow linearly with the input size (FIG. 10E), with the exception of RAM usage for ALS (FIG. 10F). Comparing NOMAD2 with STAR on the ALS dataset, NOMAD completed computations at least 1.7× faster and consumed 4× less memory (FIG. 10F). In all run-time comparisons between NOMAD2 measurements NOMAD2 was compared to the STAR alignment step alone which is typically less intensive than custom downstream bioinformatics analyses which are not required for NOMAD2.

Previous analysis of single-cell data was limited by computational and statistical implementation in NOMAD permitting parallel analysis of only 400 cells (see Example 5). However, NOMAD2 could jointly analyze 1,553 SmartSeq2 (SS2) muscle cells from donor 2 (TSP2) in Tabula Sapiens data set and show biological insights enabled by inference at this scale. NOMAD2 was run with lookahead distance 0 and concatenated anchor-target pairs, referred to as extendors, classifying anchors by the two most abundant targets per anchor as in Example 5.

NOMAD2 rapidly identifies candidate RNA editing de novo, including detecting potentially hyperedited events, filling a gap in existing bioinformatic tools. To identify anchors whose target diversity could be explained by the canonical A-to-I editing, anchors classified as “mismatch” were defined as cases where the two most abundant targets differ by single-base mismatches, has at least four uniquely mapping extendors to T2T each with >5% of anchor reads. The probability of such events under a model of sequencing error is vanishingly small as each cell in the corpus is sampled from a donor and hence a common genetic background.

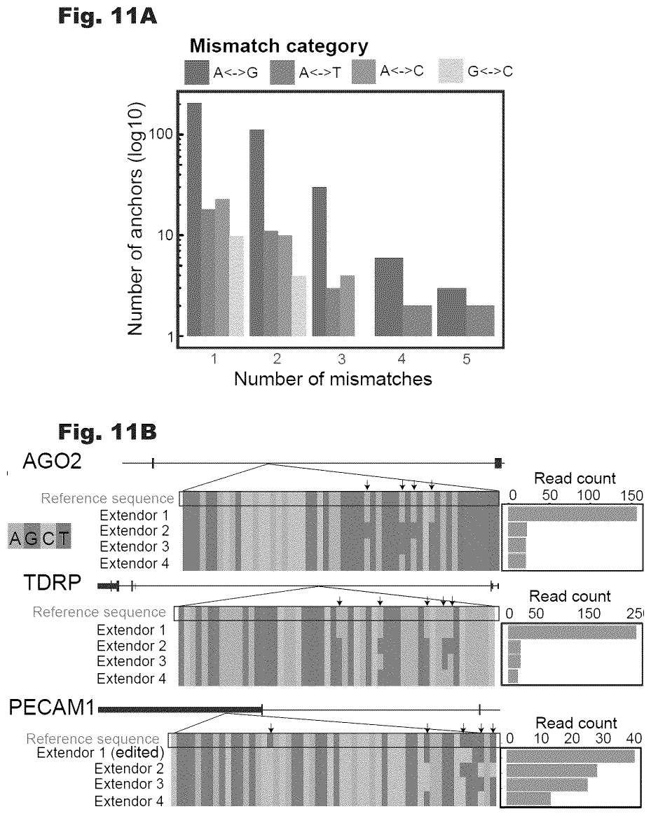

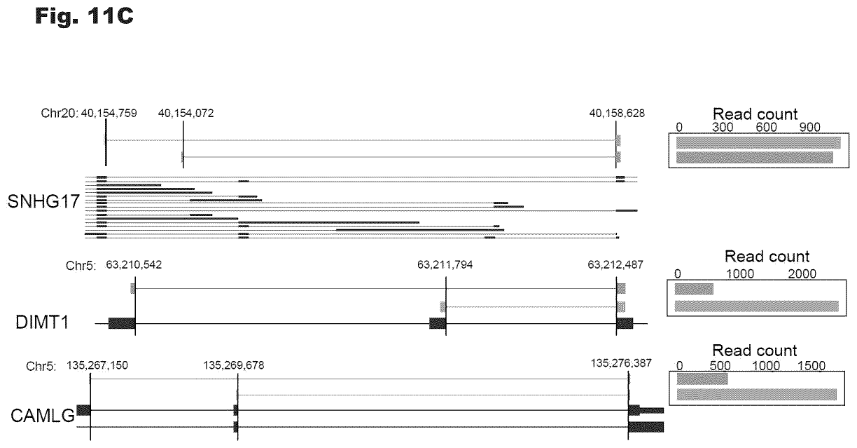

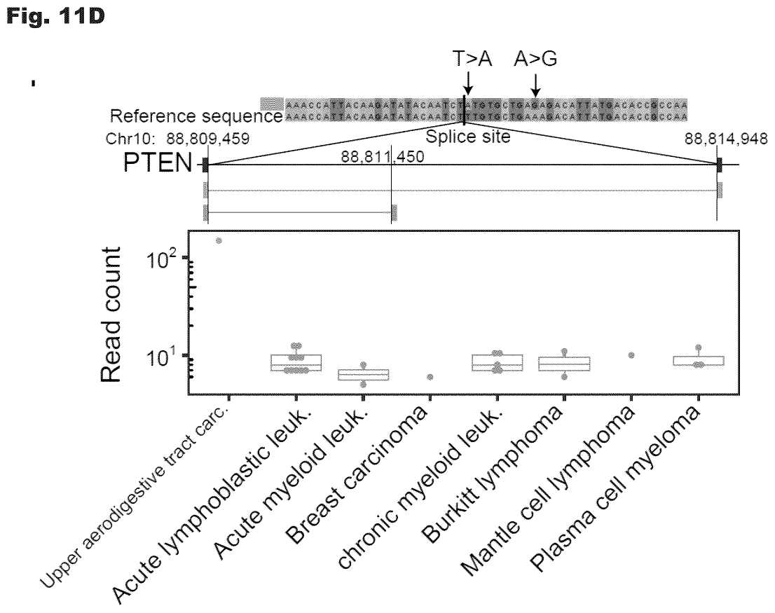

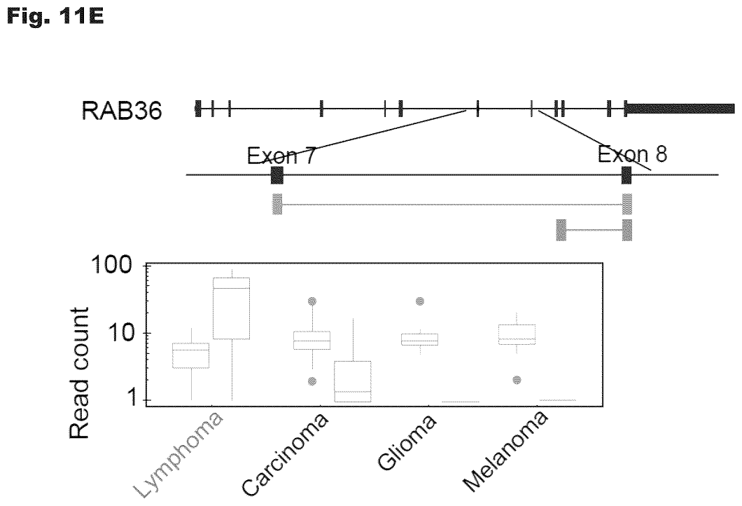

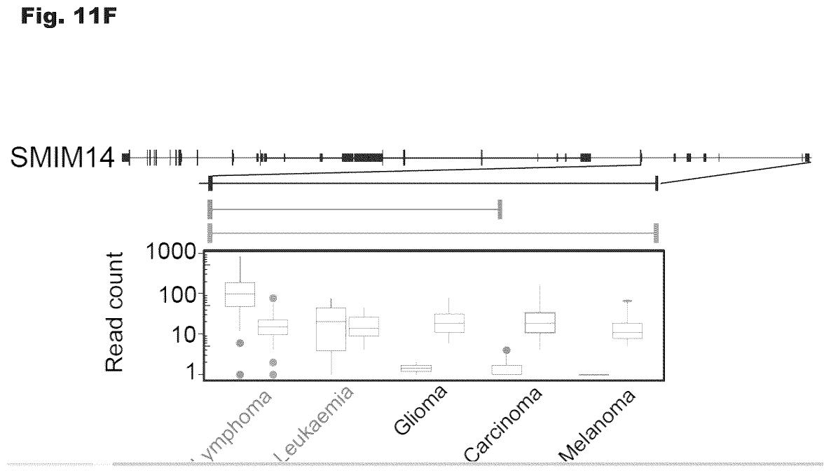

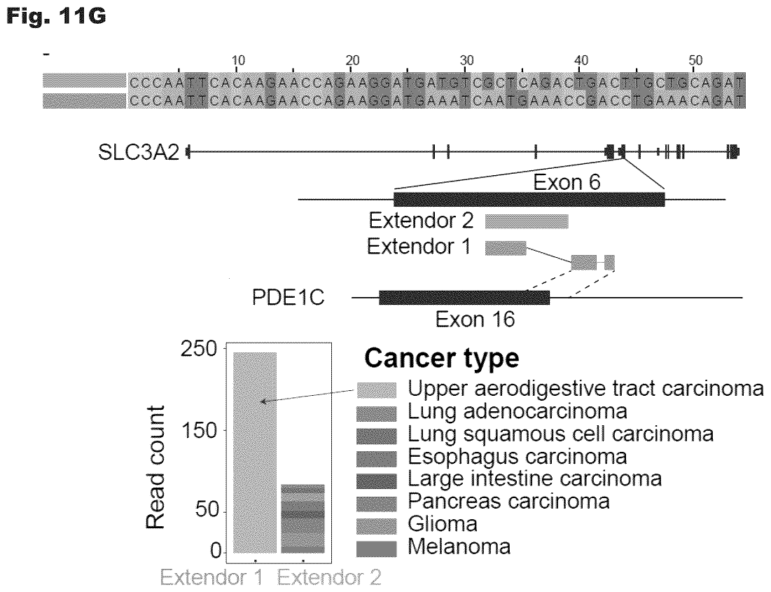

NOMAD2's analysis uncovers 512 representative anchors from 303 genes, where anchors' targets differ by single-base changes and are classified in some mismatch category (FIG. 11A). Anchors were identified where target diversity derives only from A<->G (or equivalently its reverse complement T<->C) changes. Such anchors are consistent with A-to-I editing. Similarly, anchors whose target diversity can be explained by other base changes (e.g., A<->T, G<->C) were assigned to a “mismatch” category (FIG. 11A). The A<->G category is ~10-fold more than other categories: A<->T is the next most prevalent category. Total representative anchors when the most abundant targets differ by one base pair change are: 265 (A<->G), 22 (A<->T), 19 (A<->C), and 4 (G<->C).

Anchors detected in the highest number of cells and categorized as A<->G mismatch where dominant targets differ in multiple positions (classified as >3 mismatches) map to AGO2, GNB1, and TDRP, detected in 40 (2.5%), 41 (2.5%), and 29 (1.8%) cells, respectively (FIG. 11B). Findings in AGO2 support previous reports of editing which, like this analysis, also detected circRNA products from this anchor. All mismatches in extendors assigned to GNB1 are consistent with RNA editing based on BLAT to the T2T genome (BLAT) however have ambiguous mapping in hg38. The dominant target for GNB1 had one position and the second, two positions consistent with editing. Five mismatch positions were found (including one annotated SNP according to dbSNP155) classified as G<->A mismatched, in 3′ UTR of PECAM1, a regulator of endothelial junctional integrity, where in three positions targets diverge consistent with A-to-I editing and cannot speculate about a causative mechanism for the other positions (FIG. 11B).

NOMAD2 also enables detection of annotated and novel alternative splicing. STAR alignment was used on NOMAD2 extendors to classify unannotated alternative splicing events defined by the junctional coordinates assigned by the two most abundant extendors. Anchors are classified as unannotated if at least one STAR-mapped junction was not annotated in the T2T human reference genome. NOMAD2 found 13,786 and 11,584 cell-regulated annotated and unannotated respectively in SS2 muscle cells. Anchors classified as unannotated alternative splicing and found in the highest number of cells were in SNHG17, a noncoding RNA with reported roles in tumorigenesis, and predicted to change the protein coding in CAMLG, a calcium-modulating cyclophilin ligand, and DIMT1, an RNA-modifying enzyme with a role in ribosome biogenesis in 100 and 109 cells, respectively (FIG. 11C).

To further showcase NOMAD2's discovery power, unannotated alternative splicing was analyzed in the entire 671 cell lines (5.7 TB) from primary tumors in Cancer Cell Line Encyclopedia (CCLE), a comprehensive transcriptomic and phenotypic atlas, widely used for functional cancer biology (M. Ghandi, et al., (2019), cited supra). NOMAD2 took ~47 hours with 50 Gb memory (compared to ~80 hours with STAR using the same computing resources and memory and ~4 TB of storage needed for BAM files). Separate algorithms have been used to profile mutations, splicing, and other events. However, these alignment-first analyses are expected to suffer from poor sensitivity to unannotated splicing especially in cases where there are mutations in exonic sequence, high rates of mutation, or mapping to repetitive regions that are well-known sources of mapping failure. Yet, all of these events are critical to tumorigenesis and are likely to provide potentially excellent biomarkers and neoantigens.

Accumulating evidence has shown many tumors acquire mutations in the spliceosome, and that activation of cryptic alternative splicing programs contributes to pathology. Thus, it is of great importance to precisely detect cryptic splicing in tumors. This problem is especially difficult because tumors also have a high mutational burden and accumulate structural variants missing from the reference genome, some of which are extensive. NOMAD2 was used to detect splicing events that were differentially regulated by cell line in the CCLE. NOMAD2 detected 6,697 genes categorized as being called due to differential alternative splicing; 5,755 genes had at least one unannotated alternative splicing (with >10% of reads). Previous analysis of CCLE determined that MDM4, a p53 regulator, had cell-line-specific alternative splicing. NOMAD2 recovers this positive control, skipping of exon 6, as well as detecting a novel junction not previously reported.

199 out of 5,755 genes for which NOMAD2 found unannotated alternative splicing in CCLE were cataloged in COSMIC database (J. G. Tate, et al., Nucleic Acids Research 47 (D1): D941-47 (2019), the disclosure of which is incorporated herein by reference), a database of genes with known roles in tumorigenesis, implying an enrichment of COSMIC genes in NOMAD2 calls (exact binomial p-value=1.92e-6, Methods). COSMIC genes CTNNA2, NSD2, PTEN, and COL1A1 were the genes with the highest effect size among NOMAD calls with unannotated alternative splicing. PTEN is a classical tumor suppressor. Loss of PTEN has been linked to resistance to T cell immunotherapy. In addition, alternative splice variants of PTEN have been shown to phenocopy PTEN loss. One extendor for this gene represents novel splicing between exon 3 and a cryptic exon in the next intron (24 cell lines) and the other extendor represents annotated splicing between exon 3 and 4 but with two mismatches, one internal A to G in exon 4 and a T to A base pair change at the first base of exon 4 (FIG. 11D). This establishes NOMAD's unique statistical power to jointly detect splicing and multiple genome mismatches to provide potentially new markers of PTEN loss, and more generally identify regulated splicing and sequence variation missing from the reference.

A cancer-type-specific cryptic exon of RAB36 was detected in lymphoma cell lines (21/22 lymphoma cell lines with expression for the anchor) (FIG. 11E). RAB36 is a member of the RAS oncogene family and previous studies have shown significant association between the overexpression of this gene and poorer prognosis in leukemia. Similarly, NOMAD2 detected a splicing specific to leukemia and lymphoma (21 and 15 cell lines, respectively) in SMIM14, a small integral membrane protein (FIG. 11F). This junction is not part of the T2T CAT+Liftoff annotation but was included in the NCBI refseq.

NOMAD2 discoveries extend to non-canonical transcriptional aberrations missed by STAR. To illustrate this, anchors with high effect size were considered where one extendor failed to be mapped by STAR but the other extendor could be assigned to a gene. An anchor was found in which the unmapped extendor had 75% of total anchor reads and the other extendor mapped to SLC3A2 (FIG. 11G). BLAT shows high expression of a sequence from PDE1C inserted into exon 5 SLC3A2 in one upper aerodigestive tract carcinoma cell line (FIG. 11G). The PDE1C enzyme is thought to influence pathological vascular remodeling by regulating the stability of growth factor receptors. SLC3A2 has previously been reported in a recurrent gene fusion in primary tumors, though the event detected in CCLE has not yet been reported.

Finally, NOMAD could not complete on the ALS data which profiles the effect of loss of the critical RNA binding protein TDP-43 from the nucleus in ALS. NOMAD2 extendors detect cryptic splicing in positive controls UNC13A, STMN2, and POLDIP3 recently experimentally validated to drive progression of the disease. Beyond these examples, NOMAD2 predicted novel cryptic splicing events missed by other methods.

In summary, NOMAD is a highly efficient, general analytic framework for biological discovery. In addition to improving performance of common specialized bioinformatic tools, it discovers novel mutations, transcribed structural variation and post-transcriptional regulation that may be completely missed by alignment-based methods. To date, the scale of this missing variation has been difficult or impossible to measure in large scale studies, and may be extensive in tumors. NOMAD2's scale enables a next-generation of massive genomic data analysis, including for essentially any RNA or DNA sequencing project.

### Anchors Filtering

To lower the memory requirements and computation time, several filters are applied in NOMAD2 to remove potential artifacts. The first pair of filters is applied in the first stage to discard all the anchors for a given sample where: a) its total count is not greater than 5, or b) the anchor contains a poly(A/C/G/T) string of length at least 8. In the second stage, all the anchors fulfilling at least one of the following conditions are removed: a) the sum of counts across all samples and targets is not greater than 50, b) the number of unique targets is not greater than 1, or c) the number of unique samples is not greater than 1. All the given values are defaults and can be redefined by the user. In some cases, the number of targets for a single anchor may be large, while only the most abundant ones are relevant. NOMAD2 allows keeping only a user-defined number of most frequent targets per anchor (by default all targets are used).

### Intermediate Files Format: SATC

The pipeline is implemented as a set of separate applications. For communication they store and load files in a SATC format. Each SATC file stores a list of records containing Sample id Anchor Target Count. SATC file starts with a header, which defines the number of bytes used to represent each record part, and also some other metadata. Records follow the header. The order of fields is as follows: sample id, anchor, target, count. Conceptually, for each field of a record, a minimal number of bytes is used, however, some additional techniques are applied to reduce the size of these files. After the first stage, each SATC file contains a single sample id and is sorted w.r.t. anchor, and in a range of the same anchor w.r.t. targets. In consequence, adjacent records share some number of most significant bytes. (At least the number of bytes used to represent sample id, but in many cases also the next bytes are identical.) For this reason, one additional byte to each was recorded. It contains a number of identical leading bytes with the previous record, then the identical bytes are not stored. During the reading, they are reconstructed based on the previous record. On top of this technique, zstd was also applied, a general-purpose compression algorithm to reduce the file sizes even more.

### Performance Evaluation Details

For the SS2 Muscle, KMC was run in RAM-only mode and 16 processes were assigned to the first stage (2 threads for each process), while for the ALS KMC was run in the default, disk mode, its RAM limit was set to 6 GB, and 2 processes were used in the first stage (each with 16 threads). For the second stage, 32 computing processes were used.

Increasing gap length slightly decreases all the measured values due to the lower number of existing (a+g+t)-mers: In SS2 Muscle (>1553 FASTQ files of average size 657 MB), the most resource-demanding is the second stage, in the case of over a dozen large input samples (ALS) the first stage is roughly 74% of the total computing time.

To measure the impact of the input data size NOMAD2 was run on a number of subsets of SS2 Muscle samples (FIG. 10E) and also on all samples of ALS subsampled to a given number of reads (FIG. 10F). In both cases, running time grows linearly with the input size. In the case of SS2 Muscle, the memory usage also grows linearly, yet it is negligible in comparison with the input size. In the case of ALS, the memory grows with the number of input samples up to about 300 M reads and then it saturates. STAR was run with 32 threads, one sample at a time.

### Merging SATC Files

In the second stage, for a single process, there are as many input SATC files, as the samples. Depending on the dataset, there may be hundreds of input samples. To merge them efficiently, a binary heap was used. For each unique anchor, a min-heap of size equal to the number of input samples was maintained. Another binary heap is used for filtering out the least frequent targets. This is only performed if the user defined the maximal number of most frequent target, nt to be used for contingency table construction.

### The Architecture of the Code and Parallelization Guidance

NOMAD2 is composed of a set of applications written in the C++ programming language accompanied by a single Python script to run them as separate processes. In the first stage, KMC and KMC-tools were used, which are parallel themselves. All the other binaries are single-threaded. In particular, the binary that merges bins and computes statistics in the second stage is sequential. To run NOMAD2 using p cores it is recommended to configure the number of processes for the second stage to p. On the first stage the decision should be based on the number of input samples. If there are no more than ten input samples, it is recommended to run a small number of first stage processes and assign more threads to each such process (for example for p=32 use 4 processes, each with 8 threads). In a case of a hundred (or more) input samples, it may be more beneficial to run more processes (for example 16 processes with 2 internal threads). Lower number of processes will usually lead to lower memory consumption.

### Statistical Methodology

By default, NOMAD2 computes several measures for interpretability and processing of anchors, such as average target Hamming distance, average target edit distance, and target entropy. The optimization procedure for the p-value computation identifies latent structure in the matrix by splitting the counts matrix into train and test portions, computing 1-dimensional row and column embeddings (f and c) respectively (original problem is bi-convex so this is performed via alternating maximization). These f and c are then used, with the held-out test data, to yield a statistically valid p-value, and compute an effect size measure, which indicates how well the samples can be separated into two groups. Additional outputs such as an asymptotically-valid p-value are also available.

### Splicing Anchors

A post facto step was performed to find anchors whose target sequence diversity is due to alternative splicing. For each pair of anchor and target sequence, their sequences were concatenated and then aligned to the reference genome using STAR. An anchor is classified as a splicing anchor, if STAR reports a splice junction for at least one of the sequences corresponding to its top two most abundant targets.

### RNA Mismatch Classification

If all the base pair substitutions between the targets of an anchor can be performed by a distinct mapping, a mismatch category was assigned to the anchor. For example, a category “mismatch G<->A” was assigned to an anchor, if all its targets' sequences reduce to the same sequence when all G's in target sequences are changed to A. 6 different categories were assigned: G<->A, T<->C, T<->A, C<->A, G<->C, and T<->G. Note that some of these categories are reverse complements of each other such as G<->A with T<->C and C<->A to T<->G, that can be collapsed when data is not strand specific.

To compute the probability of observing 4 targets>5% abundance, under a biallelic mode, the following model was used. In a diploid genome, there are two alleles, and thus observing non-modified or edited RNA where the anchor is adjacent to a bi-allelic SNP should result in observing two targets with a high abundance. To compute the probability of observing 4 targets each with >5% abundance, this probability was bound by the probability that either allele has a second target of >5% abundance. It was assumed that there is only 1 allele and compute the probability of observing a target with >5% abundance. There are 27*3 number of different 1 base pair changes from the dominant target which was assumed to be an un-modified allele. The first order approximation of the probability of an observed target differing from the dominant allele is that the target has one mismatch and if sequencing error is 1%, the probability of observing this target is less than 0.01/3. It was then computed, with n.trials=27*3 to estimate the probability of observing one target of >5% abundance of all anchor counts as follows:

$$> sum ( rbinom ( n . trials , N , ( \text{.01} / 3 ) ) / N > \text{.05} / n . trials$$

Where N # is the observations per anchor eg counts. Setting N=100 to 1000 yields (up to computational precision) a value of 0.

### Enrichment Analysis of COSMIC Genes

To compute the p-value for the enrichment of COSMIC genes in genes with unannotated alternative splicing in CCLE, the number of unique gene names with detected significant anchor in CCLE were counted, which was 40,133. Using this as the number of background genes, the null probability of a NOMAD-called gene being a COSMIC gene is 736/40,133=0.0183. Considering this null probability, the exact binomial p-value for the enrichment of COSMIC genes in genes with unannotated alternative splicing is <2.2e-16. However, to be more conservative 30,000 was considered as the number of background genes reflecting the approximate number of protein coding genes which results in null probability of 0.024 and exact binomial p-value of 1.92e-6.

What is claimed is:

1. A computational method for detecting nucleic acid diversity in a nucleic acid sequencing result, comprising:
retrieving a nucleic acid sequencing result of a plurality of nucleic acid samples utilizing a computational processing system, wherein the nucleic acid sequencing result comprises a set of sequencing reads for each nucleic acid sample;
calling a set of anchor sequences utilizing the computational processing system, wherein each anchor sequence of the set is a contiguous sequence that is homologous across a subset of sequencing reads of at least one nucleic acid sample;
identifying a target sequence that is downstream at a defined distance from the anchor sequence of its sequencing read for each sequencing read of the subset of sequencing reads for each anchor sequence utilizing the computational processing system;
counting a number of unique anchor sequences and the number of targets for each unique anchor sequence for each sample utilizing the computational processing system;
determining a nucleic acid sequence diversity for each sample utilizing the computational processing system, wherein the nucleic acid sequence diversity for each sample is determined utilizing the number of unique anchor sequences and the number of target sequences for each unique anchor sequence within a statistical test that is performed with a closed-form solution to calculate p-values to find anchors with significant sample-dependent target count distribution.

2. The method of claim 1, wherein each anchor sequence is defined as a sequence a where, given observing a in a sequence read, the conditional distribution of observing a target sequence t a distance R downstream of a is sample-dependent.

3. The method of claim 1, wherein each anchor sequence is set to a certain number of nucleotides.

4. The method of claim 3, wherein the certain number of nucleotides is between 3 nucleotides and 10 nucleotides.

5. The method of claim 1, wherein each anchor sequence is an adjacent, disjoint sequence.

6. The method of claim 1, wherein each anchor sequence is a tiled sequence that begins at a fixed step size.

7. The method of claim 1, wherein calling a set of anchor sequences comprises calling only anchors having a total count greater than threshold across all samples.

8. The method of claim 7, wherein the threshold is 50 total counts.

9. The method of claim 1, wherein calling a set of anchor sequences comprises discarding anchors that only appear in a number of samples below a threshold.

10. The method of claim 9, wherein the threshold is 2 samples.

11. The method of claim 1, wherein calling a set of anchor sequences comprises discarding anchor and sample pairs in which a number of sequence reads with anchors within the sample is below a threshold.

12. The method of claim 9, wherein the threshold is 6 reads with an anchor within the sample.

13. The method of claim 1, wherein the sequencing reads for each sample comprises at least 1,000 sequence reads.

14. The method of claim 13, wherein calling the set of anchor sequences comprises analyzing each sequencing read of each sample.

15. The method of claim 1, wherein each sequencing anchor of the set is a contiguous sequence that is homologous across a subset of sequencing reads of at least two nucleic acid samples.

16. The method of claim 1 further comprising sequencing the plurality of nucleic acid samples to yield the nucleic acid sequencing result utilizing a high-throughput sequencer.

17. The method of claim 1, wherein the target sequences for each unique anchor sequence is utilized to generate a consensus target sequence.

18. The method of claim 1, wherein the target sequences for each unique anchor sequence is utilized to group target sequences via a homology branching method.

19. The method of claim 1, wherein the target sequences for each unique anchor sequence is utilized to lexicographically sort the target sequences.

20. The method of claim 1 further comprising classifying at least one unique anchor sequence based on a sequence divergence of its target sequences utilizing the computational processing system.

21. The method of claim 20, wherein the classification is one of: nucleotide polymorphism, classical splice site, internal splice site, 3′UTR, centromere, or repeat.

22. The method of claim 1, further comprising aligning at least one unique anchor sequence with a reference to provide context utilizing the computational processing system.

23. The method of claim 1, further comprising decoding at least one unique anchor sequence to yield an amino acid sequence utilizing the computational processing system.

24. The method of claim 1, wherein the plurality of nucleic acid samples comprises one of: a viral genome sample for assessing viral strains, a microbiome sample for assessing microbial genera or species diversity, a cancer sample for assessing clonal diversity, an expressed transcript sample for assessing paralogous gene expression, an expressed transcript sample for assessing alternative splicing events, an immune cell sample for assessing adaptive immunity recombination, or a genomic sample for assessing transposon activity.


SEQUENCE LISTING (6 sequences)

SEQ ID NO: 1 - DNA, 27 bp, synthetic construct
  source 1..27: mol_type = other DNA
cctcagaggagggcgggaacagcgtga

SEQ ID NO: 2 - DNA, 27 bp, synthetic construct
  source 1..27: mol_type = other DNA
ctcggtcactctgttcccgccctcctc

SEQ ID NO: 3 - DNA, 27 bp, synthetic construct
  source 1..27: mol_type = other DNA
gccccctcggtcactctgttcccgccc

SEQ ID NO: 4 - DNA, 27 bp, synthetic construct
  source 1..27: mol_type = other DNA
gggcgggaacagcgtgaccgagggggc

SEQ ID NO: 5 - DNA, 27 bp, synthetic construct
  source 1..27: mol_type = other DNA
tcactctgttcccgccctcctctgagg

SEQ ID NO: 6 - DNA, 10 bp, synthetic construct
  source 1..10: mol_type = other DNA
ccattccatt